SEQUENCE LISTING (8 sequences)

SEQ ID NO: 1 - DNA, 26 bp, Homosapiens
agggttagggttagggttagggttag

SEQ ID NO: 2 - DNA, 18 bp, Homosapiens
aatccgtcgagcagagtt

SEQ ID NO: 3 - DNA, 44 bp, Homosapiens
aatccgtcgagcagagttagggttagggttagggttagggttag

SEQ ID NO: 4 - DNA, 6 bp, Homosapiens
ttaggg

SEQ ID NO: 5 - DNA, 38 bp, Homosapiens
ttttttaatccgtcgagcagagttagggttagggttag

SEQ ID NO: 6 - DNA, 24 bp, Homosapiens
ttttttaatccgtcgagcagagtt

SEQ ID NO: 7 - DNA, 38 bp, Homosapiens; identical to SEQ ID NO: 5

SEQ ID NO: 8 - DNA, 50 bp, Homosapiens
ttttttaatccgtcgagcagagttagggttagggttagggttagggttag

(19) United States
(12) Patent Application Publication
(10) Pub. No.: US 2010/0261162 A1
(43) Pub. Date: Oct. 14, 2010

(54) Methods of assaying for telomerase activity and compositions related to same

(76) Inventors: Edouard Collins Nice, Docklands (AU); Julie Ann Rothacker, Docklands (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/644,197

(22) Filed: Dec. 22, 2006

Related U.S. Application Data
(60) Provisional application No. 60/764,183, filed on Jan. 31, 2006.

(30) Foreign Application Priority Data
Dec. 23, 2005 (AU) 2005907287

(51) Int. Cl. C12Q 1/68

(52) U.S. Cl. 435/6

(57) ABSTRACT

The present invention relates generally to the field of diagnostic and prognostic assays such as diagnostic assays for conditions associated with telomerase activity. More particularly, the present invention provides an assay for measuring telomerase activity as an indicator of cancer, an inflammatory disorder and/or a condition involving embryogenesis and/or requiring stem cell proliferation and agents and kits useful for same. Automated and partially automated assays permitting high throughput screening also form part of the present invention. The subject invention further contemplates methods of treatment using agents identified by the subject assay or where treatment protocols are monitored by the assay.

### CROSS-REFERENCE TO RELATED APPLICATIONS

This application claims the benefit of Australian Patent Application No. 2005907287, filed Dec. 23, 2005 and U.S. Application No. 60/764,183, filed Jan. 31, 1006, each of which are hereby incorporated by reference in their entirety.

### FIELD OF THE INVENTION

The present invention relates generally to the field of diagnostic and prognostic assays such as diagnostic assays for conditions associated with telomerase activity. More particularly, the present invention provides an assay for measuring telomerase activity as an indicator of cancer, an inflammatory disorder and/or a condition involving embryogenesis and/or requiring stem cell proliferation and agents and kits useful for same. Automated and partially automated assays permitting high throughput screening also form part of the present invention. The subject invention further contemplates methods of treatment using agents identified by the subject assay or where treatment protocols are monitored by the assay.

### DESCRIPTION OF THE PRIOR ART

Telomeres are repeating DNA sequences consisting of tandem GT-rich repeats, represented as (TTAGGG)ₙ located at the 3′ end of chromosomal DNA. Gradual telomere erosion occurs during normal mitotic processes due to the loss of from about 50 to 200 nucleotides of telomeric sequence per cell division ultimately resulting in cellular senescence. Telomeres protect chromosomes from fusion and degradation through the action of telomerase which is a unique reverse transcriptase that elongates teleomeric DNA (Shay et al, Hum. Mole. Gen. 10:667-685, 2001). Telomerase is relatively abundant in germline and embryonic tissues, inflammatory cells, proliferative cells of renewal tissues, as well as cancer cells. In contrast, telomerase activity is difficult to detect in normal somatic human tissues. The correlation of telomerase activity and cellular replication has prompted the association of telomerase and cancer. Telomerase activity has been found in almost all human tumors but not in adjacent normal cells (Kim et al, Science 266:2011-2015, 1994). In fact, telomerase is activated in approximately 85% of human cancers (Hiyama et al, Cancer Lett. 194:221-223, 2003). Thus, it has been proposed that upregulation or re-expression of telomerase may be a critical event responsible for continuous tumor cell growth.

Given the association of telomerase activity with diseases of cellular proliferation, including cancer, the detection of telomerase activity is of diagnostic value. Several analytical procedures for the quantification of telomerase activity have been reported. The most frequently utilized assay is Telomeric Repeat Amplification Protocol (TRAP) which is a two stage PCR-based assay. In the first stage, telomerase adds 5′-TTAGGG-3′ repeats to the end of a synthetic primer. In the second stage, the extended oligonucleotide products are amplified using a reverse primer. When visualized by autoradiography, a positive test by TRAP shows a ladder of bands. The band volume can then be quantified (Hess et al, Clin. Chem. 48:18-24, 2002). TRAP is time consuming, labor intensive, PCR-dependent and susceptible to inhibition by extracts of clinical samples. Furthermore, it is difficult to quantify telomerase activity because of logarithmic amplification of telomerase products in the PCR amplification step. The susceptibility of the TRAP assay to Taq-polymerase inhibitors often results in the production of false positive and false negative results (Weizmann et al, Chem. Bio. 5:943-948, 2004).

A similar telomerase assay that replaced the electrophoretic step of the TRAP assay with an ELISA detection system has been developed. This system is also PCR-dependent although the ELISA detection method appears to offer no clear advantage over the traditional TRAP. In an effort to eliminate technical issues associated with TRAP, in situ hybridization assays for the quantification of human Telomerase (hTR) RNA and human Telomerase Reverse Transcriptase (hTERT) mRNA were developed. However, hTR and hTERT expression does not necessarily equate to telomerase activity (Hess et al, 2002 supra).

Another telomerase assay is disclosed in PCT/IL01/00808 (WO 02/20838). This assay uses rotating quinone-functionalized magnetic beads to generate H₂O₂ within the assay. The endogenous production of H₂O₂ putatively overcomes the problem of luminol being sparingly soluble in aqueous buffer solutions. However, the rotating magnetic beads reduces the ability to develop high through put screening protocols and may impact on the sensitivity depending on the length of oligonucleotide primer employed.

Accordingly, there is a need for a reliable, sensitive and cost effective assay for the detection of telomerase activity in clinical samples which would have diagnostic, prognostic and therapeutic value for cancer, inflammatory disorders and conditions involving embryogenesis and/or in monitoring the potential for stem cells to proliferate. The assay of the present invention is applicable to human and mammalian vertebrates in non-mammalian vertebrates and plants.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide sequences are referred to by a sequence identifier number [SEQ ID NO]. The SEQ ID NOs correspond numerically to the sequence identifiers <400>1 [SEQ ID NO:1], <400>2 [SEQ ID NO:2], etc. A summary of the sequence identifiers is provided in Table 1. A sequence listing is provided after the claims.

The present invention contemplates an assay for telomerase activity in cells which provides a diagnostic and prognostic indicator of the presence of cancer cells as well as inflammatory disorders and conditions involving embryogenesis and/or for monitoring the potential for stem cells to proliferate. The assay is also useful for assessing medical treatment protocols for humans and for screening for agents which modulate telomerase activity or levels. Telomerase activity in non-mammalian vertebrates and plants may also have diagnostic value or as a research tool. In relation to vertebrates, the level of telomerase activity correlates with the presence of certain types of cells such as cancer cells as well as changes in cell physiology or proliferative potential with age and/or in response to a treatment protocol. Similarly, the levels of, or changes in, telomerase activity provides information on inflammation including proliferation as well as conditions involving embryogenesis. The assay may be automated or semi-automated to permit high throughput screening. It is based on epithelial cell capture and lysis to detect telomerase activity. The readout is luminescence. Unlike other telomerase assays, it is not a PCR based assay.

The present invention determines, therefore, the level of telomerase activity by incorporation of a label into a telomerase-catalyzed extension nucleotide sequence.

Accordingly, one aspect of the present invention contemplates a method for detecting cells from a subject exhibiting telomerase activity, said method comprising:
i) obtaining a sample of cells from said subject, contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to detection means to read the intensity of the luminescence,
   wherein the level of intensity of luminescence compared to a negative control or a known data set provides the level of telomerase activity and the number of putative cells producing telomerase.

In relation to automation, step (iii) and in particular the addition of luminol, an enhancer and/or H₂O₂ can be added automatically by the luminescence reader.

Another aspect of the present invention provides a method for detecting cells from a subject exhibiting telomerase activity, said method comprising:
i) obtaining a sample of cells from said subject, contacting magnetic particles carrying an oligonucleotide primer comprising the sequence (XₙTTAGGYₘ)ₒ wherein:
   X is a nucleotide selected from A, T, G and C;
   Y is a nucleotide selected from A, T, G and C;
   n is 0 or 1;
   m is 0 or 1; and
   o is from about 1 to about 400;
   with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to detection means to read the intensity of the luminescence,
   wherein the level of intensity of luminescence compared to a negative control or a known data set provides the level of telomerase activity and the number of putative cells producing telomerase.

Generally, in vertebrates, n is 0, Y is G and o is from about 5 to about 30. In arthropods, n is 0, m is 0 and o is from about 1 to about 30. In plants, X is T, n is 1, Y is G, m is 1 and o is from about 1 to about 30.

The presence of telomerase activity or the level of telomerase activity compared to negative or a known data set is indicative of the number of cells which possess telomerase activity. Such cells include cancer cells, inflammatory or proliferative cells or cells involved in embryogenesis including stem cells. A “negative control” may exhibit basal levels of telomerase activity. The assay is sensitive permitting the detection of telomerase activity in as few as about 1 cell to greater than 10⁶ cells to such as from 1 to 10⁶ cells.

The present invention provides, therefore, a method for detecting cells selected from cancer cells, inflammatory or proliferative cells and embryogenic cells including stem cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to detection means to read the intensity of the luminescence,
   wherein the level of intensity of luminescence compared to a control such as a control not containing cancer, inflammatory or embryogenic cells or compared to a known data set provides the level of telomerase activity and thereby the number of cells.

The “subject” may be a human or other mammal, a non-mammalian vertebrate or a plant or other entity comprising a telomerase.

As indicated above, the assay of the present invention can be automated or employed as a single assay or a batch of assays. The step of adding luminol, an enhancer and/or H₂O₂ is conveniently automated. The present invention provides, therefore, kits comprising the reagents required to perform the assay as well as instructions for use. In addition, the assay may be conducted under multiplex conditions with multiple labels. Still further, the assay may be part of a number of assays (i.e. two or more assays) to assist in cell identification or to monitor a therapeutic protocol.

The present invention enables the quantitative detection of telomerase activity in cells by the measurement of the extent of a signal. The present invention extends, however, to the use of the subject assay to provide a qualitative detection of the presence or absence or relative level of telomerase activity. Terms such as “determination”, “determining”, “detection”, “diagnosis”, “prognosis” and “identification” are used interchangeably to refer to qualitative, semi-qualitative and qualitative detection of telomerase activity in a cell or sample of cells.

In a particular embodiment, the telomerase assay is used to detect the presence of cancer cells or to monitor the progression of cancer in a subject including monitoring cancer in the presence of a chemotherapeutic agent. A “chemotherapeutic agent” in this context includes a chemical agent as well as an immunological or antibiotic agent. A “cancer” is regarded the same as a tumor as far as the present invention is concerned.

Accordingly, the present invention contemplates a method for detecting cancer cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
(iv) subjecting the resulting mixture detection means to read the intensity of the luminescence,
   wherein the level of intensity of luminescence compared to a control such as not containing cancerous cells or a known data set provides the level of telomerase activity and the presence of or number of putative cancer cells.

The present invention further extends to use of the assay to assess the efficacy of a cytotoxic agent such as an anti-cancer chemotherapeutic agent. It can also be used for risk stratification of cancer patients such as leukemia patients.

In one embodiment, “obtaining a sample of cells” includes collecting and partially purifying the cells or at least removing unnecessary components in the samples. An aspect of the present invention provides a method for selective purification of the tumor cells and removal of those cells from potentially interfering substances. Purification of the tumor cells is achieved by incubation of the body fluid containing the cells with magnetic beads, which are coated with tumor cell-specific antibody. The tumor cells of interest are washed extensively and therefore separated from other cell types, the body fluid matrix (e.g.; urine, blood), and interfering substances. This lessens the possibility of false negatives due to interference with the assay and also false positives caused by non-tumor cells such as activated T-lymphocytes which may be present in an infection. The sample workup procedure is thus considered useful in obtaining high clinical sensitivity and specificity values.

Accordingly, another aspect of the present invention is directed to a method for assessing the activity of a cytotoxic agent, said method comprising:
i) adding a putative cytotoxic agent to a culture of cancer cells;
ii) contacting magnetic particles carrying an oligonucleotide primer which is a substrate for telomerase with a cellular extract from the cancer cells and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer; iii) contacting the magnetic particles with streptavidin-horseradish peroxidase;
iv) collecting the beads using a non-rotating magnet, washing the beads and contacting the washed beads with luminol and an enhancer and exogenous H₂O₂ to generate luminescence; and
v) subjecting the resulting mixture to detection means to read the intensity of luminescence,
   wherein the level of intensity of luminescence in the presence of the cytotoxic agent compared to a control such as not containing a cytotoxic agent provides the level of cytotoxicity of the agent.

The present invention further contemplates a method of treatment using a cytotoxic agent identified using the method defined above. The method of treatment may also involve assessing a clinical protocol using the subject assay. The protocol may be varied depending on how the telomerase levels vary over time with the protocol.

The TBT can also be used to assess aging and to monitor deterioration or degree of health in elderly subjects.

The oligonucleotide primer may be immobilized to the beads by any coupling chemistry including via thiol, amine and aldehyde coupling chemistries.

In one embodiment, the oligonucleotide primer which is the substrate of telomerase is immobilized to the beads via a thiol linkage. For example, a suitable linker is represented in SEQ ID NO:5.

The telomerase assay of the present invention is referred to herein as the “TBT” or “telomerase biosensor technology”.

The method of the present invention includes the proviso that elongation of the telomerase substrate oligonucleotide primer is not via PCR.

A summary of sequence identifiers used throughout the subject specification is provided in Table 1.

TABLE 1
Summary of sequence identifiers

| SEQUENCE ID NO: | DESCRIPTION |
|---|---|
| 1 | Human Telomerase recognition nucleotide |
| | sequence |
| 2 | Magnetic bead surface-linked synthetic spacer |
| | nucleotide sequence |
| 3 | Combined surface linked spacer sequence |
| | and telomerase recognition sequence |
| 4 | Repeating nucleotide sequence added by |
| | telomerase to telomerase recognition sequence |
| 5 | Target sequence for telomerase, with a |
| | 5″ cysteine for thiol coupling |
| 6 | Short human telomerase recognition nucleotide |
| | sequence |
| 7 | Medium human telomerase recognition nucleotide |
| | sequence |
| 8 | Long human telomerase recognition nucleotide |
| | sequence |

A summary of the abbreviations used throughout the subject specification are provided in Table 2.

TABLE 2
Abbreviations

| ABBREVIATION | DESCRIPTION |
|---|---|
| CPG | Calcium Pectinate Gel |
| HRP | Horseradish peroxidase |
| hTERT | Telomerase Reverse Transcriptase |
| hTR | human Telomerase |
| TBT | Telomerase biosensor technology |
| TRAP | Telomeric Repeat Amplification Protocol |

### BRIEF DESCRIPTION OF THE FIGURES

The invention described in this specification will be better understood when taken in conjunction with the drawings briefly described below. Some figures contain color representations or entities. Color photographs are available from the Patentee upon request or from an appropriate Patent Office. A fee may be imposed if obtained from a Patent Office.

FIG. 1 illustrates a graphical representation showing the method of monitoring the conjugation of target sequence for telomerase to beads according to an embodiment of the present invention. A spectrophotometric method for monitoring the conjugation of an oligonucleotide suitable for extension by telomerase activity to a bead that may be subjected to collection by a magnet or other means. Free oligonucleotide has a peak absorbance around the wavelength of 260 nm while conjugated oligonucleotide has a peak absorbance around 343 nm.

FIG. 2 illustrates a graphical representation showing the sensitivity of the telomerase assay for LIM1215 cells according to an embodiment of the present invention. This shows that telomerase activity released from lysed LIM 1215 human colon cancer cells can be measured by fluorescence emitted by incorporated fluorescein bound nucleotide. A linear range of detection is apparent as determined by using 100 to 1000 lysed cells.

FIG. 3 illustrates a graphical representation showing results of telomerase assay on a superficial bladder cancer sample according to an embodiment of the present invention. This shows telomerase activity released from 1000 lysed LIM 1215 cells and cells collected from a patient with pathologically confirmed superficial bladder cancer. The bladder cancer cells were captured using EpCAM beads. Matched reactions were performed using lysates pretreated with heat to inactivate telomerase enzyme activity (HI). These data demonstrate telomerase activity using horse radish peroxidase conjugated streptavidin reacted with luminol to generate luminescence. The low background signal generated by the streptavidin alone is also shown.

FIG. 4 illustrates a graphical representation showing results of telomerase assay on an invasive bladder cancer sample according to an embodiment of the present invention. This shows telomerase activity released from 1000 lysed LIM 1215 cells and cells collected from a patient with pathologically confirmed invasive bladder cancer. The bladder cancer cells were captured using EpCAM beads. Matched reactions were performed using lysates pretreated with heat to inactivate telomerase enzyme activity (HI). These data demonstrate telomerase activity using horse radish peroxidase conjugated streptavidin reacted with luminol to generate luminescence. The low background signal generated by the streptavidin alone is also shown.

FIG. 5 illustrates a graphical representation showing telomerase activity in cells isolated from fecal samples according to an embodiment of the present invention. These data demonstrate the capacity to isolate know and predetermined colon cancer cells from a faecal sample using EpCAM beads and subsequent release of telomerase activity and measurement using luminescence.

FIG. 6 illustrates a graphical representation showing the sensitivity of the assay for HEK293T cells according to an embodiment of the present invention. These data demonstrate telomerase activity using horse radish peroxidase conjugated streptavidin reacted with luminol to generate luminescence. Averaged data are from 10 experiments performed using the manual assay format on different days within three different lysate preparations. This indicates that telomerase activity of stock cell lysates of HEK293T cells can be measured with reproducibility. A linear range of detection is apparent as determined by using the equivalent of 20 to 1250 lysed cells.

FIG. 7 illustrates a graphical representation showing the sensitivity of the assay for low levels of HEK293T cells according to an embodiment of the present invention. Averaged data are from 10 experiments performed using the manual assay format on different days with 3 different lysate preparations. This indicates that telomerase activity of stock cell lysates of HEK293T cells can be measured with reproducibility. A linear range of detection is apparent over the range of 1 to 500 lysed cells. These data demonstrate telomerase activity using horse radish peroxidase conjugated streptavidin reacted with luminol to generate luminescence. These data demonstrate that the TBT assay is sensitive at low cell concentrations.

FIG. 8 illustrates a graphical representation showing results of a statistical evaluation of the lower limit of detection of the TBT assay according to an embodiment of the present invention. Telomerase activity of stock cell lysates of HEK293T was measured with the TBT assay on 10 separate occasions on different days with 3 different lysate preparations. Dashed lines indicate one (1×SD) and 2 (2×SD) standard deviations (SD) above the mean background level (0 CE) which was determined 20 times. The top numbers illustrated within each bar on the histogram are the actual number of SD above background. “n” is the number of individual determinations used to generate the mean. These data demonstrate telomerase activity using horse radish peroxidase conjugated streptavidin reacted with luminol to generate luminescence.

FIG. 9 illustrates a graphical representation showing the intra-assay reproducibility of the TBT assay according to an embodiment of the present invention. The telomerase activity of two different concentrations of HEK293T tumor cells was measured. The level of variability, between six separate determinations at each concentration, 100 CE and 1000 CE, within the TBT assay appeared to be approximately 5.7% and 4.9% of the total signal, respectively. These data demonstrate telomerase activity using horse radish peroxidase conjugated streptavidin reacted with luminol to generate luminescence. Averaged data (mean±standard deviation) are from six replicate samples at each concentration.

FIG. 10 illustrates a graphical representation showing the inter-assay reproducibility of the TBT assay according to an embodiment of the present invention. The telomerase activity of two different concentrations of HEK293T tumor cells was measured on 10 separate occasions. The level of between assay variability at each concentration, 50 CE and 5000 CE, appeared to be 6.4% and 8.8% of the total signal respectively. These data demonstrate telomerase activity using horse radish peroxidase conjugated streptavidin reacted with luminol to generate luminescence. Averaged data (mean±standard error) are from four separate determinations for each concentration performed on different days.

FIG. 11 illustrates a graphical representation showing the specificity of the enzyme specificity of the TBT assay according to an embodiment of the present invention. The telomerase activity was determined in the human leukemia cell line TF-1 cells and TF-1 cells over expressing hTERT (human telomerase reverse transcriptase) over a broad range of concentrations. These data demonstrate telomerase activity using horse radish peroxidase conjugated streptavidin reacted with luminol to generate luminescence. The specificity of the assay is demonstrated by the greater telomerase activity found in the TF-1 cells overexpressing hTERT.

FIG. 12 illustrates a graphical representation showing the sensitivity of the TBT assay for the detection of telomerase activity in urine samples according to an embodiment of the present invention. The TBT test was used to measure telomerase activity in cells, isolated from the urine of patients, in cell lysate concentrations ranging from 0 μl to 2.5 μl of lysate. Telomerase activity was measured in urine cell lysates from three patients previously showing a positive TBT result, two of which had a high TBT result (Patients #3 and #12) and one patient having a low TBT result (Patient #31). These data indicate that less than 1 μl of cell lysate, representing less than one hundredth the total number of tumor epithelial cells in each patient sample, was sufficient to give a positive signal.

FIG. 13 illustrates a graphical representation showing the sensitivity and specificity of the TBT assay for the detection of telomerase activity in urine samples according to an embodiment of the present invention. The TBT test was used to measure telomerase activity in cells, isolated from the urine of bladder cancer patients (n=29) and normal subjects (n=12). When a ‘cut-off’ value of 1.5 (fold-change compared to no telomerase control) is used the assay has 96.6% sensitivity and 100% specificity. When the ‘cut-off’ threshold is 1.2 (dashed line) the sensitivity of the assay is 100% and there is a small increase in the false-positives.

FIG. 14 illustrates a graphical representation showing the sensitivity of the TBT assay for the detection of telomerase activity in K562 human leukemia cells according to an embodiment of the present invention. The TBT test was used to measure telomerase activity over a broad range of cell lysate concentrations up to 2500 CE. These data demonstrate telomerase activity using horse radish peroxidase conjugated streptavidin reacted with luminol to generate luminescence. The TBT assay shows a high level of sensitivity in analysis of telomerase activity in leukemia cells.

FIG. 15 illustrates a graphical representation showing the sensitivity of the TBT assay for the detection of telomerase activity in umbilical cord blood stem cells according to an embodiment of the present invention. CD34-positive cells from the cord blood of three patients were isolated and the TBT assay was performed on 1000 CD34-positive cells. Telomerase activity was detected in all three cord blood samples.

FIG. 16 illustrates a graphical representation showing the effect of TBT oligonucleotide length in HEK293T cell lysates according to an embodiment of the present invention.

FIG. 17 illustrates a graphical representation of a receiver operating characteristic (ROC) curve showing the diagnostic power of the TBT test in detecting bladder cancer according to an embodiment of the present invention.

### DETAILED DESCRIPTION OF THE INVENTION

The present invention provides a sensitive assay for telomerase in a cell or sample of cells. The test is referred to as “TBT” or “telomerase biosensor technology”. Reagents useful for conducting the assays also form part of the present invention. The reagents may be part of a kit packaged with instructions for performing the assay or may be separately provided. Detection of telomerase may be quantitative, semi-quantitative or qualitative which are all encompassed by the terms “determination”, “determining”, “detection”, “diagnosis”, “prognosis” and “identification”. The assay may be automated or semi-automated to permit rapid, high throughput screening. The elucidation of the presence of telomerase activity including the level of telomerase activity is useful for determining the presence or relative levels of cancer cells or cells associated with inflammation, proliferation and/or embryogenesis. Whilst the principle focus of the invention is in humans, the assay may be conducted in all vertebrates, plants and arthropods.

Having regard to the method and reagents employed in accordance with the present invention, it is apparent that the assay has a range of research and diagnostic applications. The assay is fast, accurate and amenable to single-tube reactions, multiplex protocols, automation and in situ detection. The use of magnetic beads enables routine clinical use at a low cost whilst maintaining high sensitivity and clinical sustainability. Other telomerase assays are expensive, cannot be modified for high throughput screening and cannot be routinely used in clinical laboratories. Applications of TBT include, but are not limited to:
i) detection of immortal cells in cancer biopsies for the identification of potential cancer cells;
ii) identification in a cell-based or cell-free screen of agents capable of activating, derepressing, inhibiting or repressing telomerase, including immortalizing agents (e.g. oncogenes) or compounds that might activate telomerase and extend telomeres and replicative lifespan of cells;
iii) identification in culture systems or in vivo of stem cells or early progenitor cells that possess telomerase activity;
iv) examination of telomerase regulation during differentiation and development;
v) identification of telomerase-positive fractions generated during purification of telomerase;
vi) identification of protozoal or fungal infections; and
vii) diagnosis of certain types of infertility characterized by an absence of telomerase activity.

The TBT is high throughput, very sensitive inexpensive, and can be routinely employed in a clinical laboratory.

Accordingly, one aspect of the present invention contemplates a method for detecting cells from a subject exhibiting telomerase activity, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to detection means to read the intensity of the luminescence,
   wherein the level of intensity of luminescence compared to a negative control or a known data set provides the level of telomerase activity and the number of putative cells producing telomerase.

In a related embodiment, the present invention contemplates a method for detecting cells from a subject exhibiting telomerase activity, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence wherein said enhancer and H₂O₂ are added automatically in a machine which measures luminescence intensity; and
iv) subjecting the resulting mixture to detection means to read the intensity of the luminescence,
   wherein the level of intensity of luminescence compared to a negative control or a known data set provides the level of telomerase activity and the number of putative cells producing telomerase.

In one embodiment, “obtaining a sample of cells” includes collecting and partially purifying the cells or at least removing unnecessary components in the samples. An aspect of the present invention provides a method for selective purification of the tumor cells and removal of those cells from potentially interfering substances. Purification of the tumor cells is achieved by incubation of the body fluid containing the cells with magnetic beads, which are coated with tumor cell-specific antibody. The tumor cells of interest are washed extensively and therefore separated from other cell types, the body fluid matrix (e.g.; urine, blood), and interfering substances. This lessens the possibility of false negatives due to interference with the assay and also false positives caused by non-tumor cells such as activated T-lymphocytes which may be present in an infection. The sample workup procedure is thus considered useful in obtaining high clinical sensitivity and specificity values.

By way of example, urine is incubated with magnetic beads coupled with monoclonal antibody, Ber-EP4 (CELLection [Trade Mark] Epithelial Enrich Dynabeads), which selectively captures the epithelial cells. The beads with tumor cells attached are washed several times and lysis of the epithelial cells achieved by addition of CHAPS-based lysis buffer. The advantage of this method is that it separates the tumor cells from potentially interfering substances and also activated lymphocytes, which may contain elevated telomerase activity.

The sample workup procedure removes the cells of interest from many chemicals that may commonly interfere with clinical assays. Isolation of the epithelial cells from blood removes any possibility of interference from hemoglobin, degradative enzymes in urine, or therapeutic compounds such as those used for chemotherapy or other treatments.

The presence of activated lymphocytes has proven problematic for other assays of telomerase activity as these cells can express detectable levels of telomerase activity. The sample workup procedure in the TBT test removes the tumor epithelial cells from activated lymphocytes by selective capture on antibody-attached magnetic beads. Removal of tumor cells from activated lymphocytes leads to greater sensitivity and a lower probability of false positives. Whilst useful, this should not be regarded as an essential feature of the present invention.

The term “subject” includes a vertebrate such as a human or non-human mammal, non-mammalian vertebrate, a plant or other entity comprising a telomerase.

As indicated above, in relation to vertebrates, the cells may be cancer cells or cells associated with inflammation, proliferation or embryogenesis. Accordingly, another aspect of the present invention provides a method for detecting cells selected from cancer cells, inflammatory or proliferative cells and embryogenic cells including stem cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to detection means to read the intensity of the luminescence,
   wherein the level of intensity of luminescence compared to a control such as a control not containing cancer, inflammatory or embryogenic cells provides the level of telomerase activity and thereby the number of cells.

In a related embodiment, the present invention provides a method for detecting cells selected from cancer cells, inflammatory or proliferative cells and embryogenic cells including stem cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence wherein said enhancer and H₂O₂ are added automatically in a machine which measures luminescence intensity; and
iv) subjecting the resulting mixture to detection means to read the intensity of the luminescence,
   wherein the level of intensity of luminescence compared to a control such as a control not containing cancer, inflammatory or embryogenic cells provides the level of telomerase activity and thereby the number of cells.

As indicated above, the terms “cancer”, “tumor” and “cancerous” may be used interchangeably throughout the subject specification and denotes any cancerous or malignant condition, pre-cancerous condition, myeloma, or any lymphoma or malignant condition, or any other proliferative disorder involving neoplastic cells. The term “cancer” or “tumor” includes breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's-cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed-cell, null-cell, t-cell, t-cell chronic, htlv-ii-associated, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nscic), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (scic), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), sertoli cell tumor, synovioma, testicular cancer, thymus cancer, thyroid cancer, transitional-cell-cancer-(bladder), transitional-cell-cancer-(renal-pelvis−/−ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

Moreover, for the purposes of the present invention, the term “a” or “an” entity refers to one or more than one of that entity; for example, “a cancer cell” or “an reagent” refers to one or more of those compounds, or at least one compound. As such, the terms “a” or “an”, “one or more” and “at least one” can be used interchangeably herein. It is also to be noted that the terms “comprising”, “including”, and “having” can be used interchangeably. Furthermore, a compound “selected from the group consisting of” refers to one or more of the compounds in the list that follows, including mixtures (i.e. combinations) of two or more of the compounds. According to the present invention, an isolated or biologically pure compound is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

The TBT is a useful assay for risk stratification of cancer patients, such as for risk of remission or cancer spread.

An inflammatory or proliferative condition includes cells associated with acne, angina, arthritis, aspiration pneumonia, disease, empyema, gastroenteritis, inflammation, intestinal flu, nec, necrotizing enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy or chronic inflammatory demyelinating polyradiculoneuropathy.

In a particular embodiment, the telomerase activity is used to quantitate, semi-quantitate or qualitate the presence or level of cancer cells. Reference to “cancer” includes a tumor and a leukemia as well as carcinoma and a sarcoma.

Accordingly, another aspect of the present invention provides a method for detecting cancer cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
(iv) subjecting the resulting mixture detection means to read the intensity of the luminescence,
   wherein the level of intensity of luminescence compared to a control such as not containing cancerous cells and/or a known data set provides the level of telomerase activity and the number of putative cancer cells.

In another particular embodiment, the present invention provides a method for detecting cancer cells in a sample from a subject, said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence wherein said enhancer and H₂O₂ are added automatically in a machine which measures luminescence intensity; and
(iv) subjecting the resulting mixture detection means to read the intensity of the luminescence,
   wherein the level of intensity of luminescence compared to a control such as not containing cancerous cells and/or a known data set provides the level of telomerase activity and the number of putative cancer cells.

Cell extracts may be generated by any number of means including sonnication, lysis and freeze-thaw methods. In one embodiment, cells are collected by biopsy or in a blood or tissue sample, and lysed using a non-ionic and/or zwitterionic detergent. Cell debris is generally removed by centrifugation or filtration. The supernatant is then collected and used in the assay. Examples of suitable detergents include Tween 20, Triton X-100, Triton X-114, Thesit, NP-40, n-octylglucoside, n-dodecylglucoside, n-dodecyl-beta-D-maltoside, octanoyl-N-methylglucamide (MEGA-8), decanoyl-N-methylglucamide (MEGA-10), and isotridecylpoly(ethyleneglycolether)ₙ, and preferred zwitterionic detergents include CHAPS (3-{(3-cholamidopropyl)dimethylammonio}-1-propane-sulfonate), CHAPSO (3-{(3-cholamidopropyl)dimethyl-ammonio}-2-hydroxy-1-propane-sulfonate), N-dodecyl-N,N-dimethyl-3-ammonio-1-propane-sulfonate, and digitonin, with CHAPS.

In another particular embodiment, the cells are lysed with CHAPS buffer [0.5% v/v CHAPS, 10 mM Tris, 1 mM MgCl₂, 1 mM EGTA and 10% v/v glycerol with 1 protease inhibitor tablet (Compete Mini, Roche) per 10 ml].

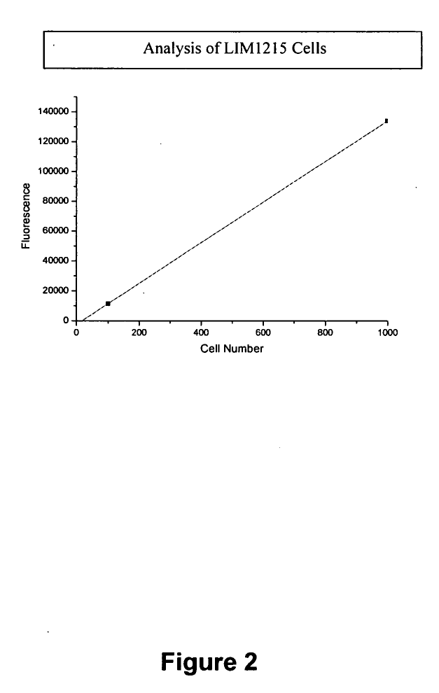

Cell collection may be by any means and numbers of cells in a sample to be assayed may vary. Generally from about 1 cell to about 10⁶ or greater cells may be assayed at a time. Hence, the present invention is capable of assaying from 1 to 10¹⁰ cells including 5 to 10⁶ cells, 10 to 10⁵ cells and so on. Particularly useful cell numbers include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 or 1000 cells or 5×10³, 10⁴, 5×10⁴, 10⁵, 5×10⁵, 10⁶, 5×10⁶ and 10⁷ cells. The sensitivity of the assay can be seen from FIG. 2. In a particularly convenient aspect of the method, cell samples are not diluted prior to the assay but rather volumes of cells are removed to provide from about 1 cell to 10⁶ cells or greater.

Hence, a sensitivity of from 1 to 1000 cells is particularly preferred such as measuring 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999 or 1000 cells in a sample.

Any type of magnetic particle may be employed in the practice of the assay of the present invention. Typically, the particles are made from Fe₃O₄, Fe, Co, Ni, their alloys as well as other ferromagnetic materials. Although not wishing to limit the present invention to any type of bead, Dynal (trademark—Dynal Invitrogen Corporation, 9099 North Deerbrook Trail, Brown Deer, Wis., USA 53223) or Bioclone (San Diego, Calif. 92126, USA) beads or CPG calcium pectinate gel magnetic beads (CPG Inc, Lincoln Pk, N.J. 07035, USA) may be employed. Lode Star, polymer-based beads (Polymer Labs, UK and USA) may also be employed.

The telomerase substrate, i.e. the oligonucleotide primer, comprises the sequence:

(XₙTTAGGYₘ)ₒ

wherein:
X is selected from A, T, G and C;
Y is selected from A, T, G and C;
n is 0 or 1;
m is 0 or 1; and
o is from 1 to about 400.

Generally, in vertebrates, n is 0, Y is G and o is from about 5 to about 30. In arthropods, n is 0, m is 0 and o is from about 1 to 30. In plants, X is T, n is 1, Y is G, m is 1 and o is from 1 to about 30.

In one particular embodiment, the magnetic beads comprise a human telomerase target nucleotide sequence [SEQ ID NO:1] immobilized to their surface. The human telomeric target sequence is 5′-AGGGTTAGGGTTAGGGTTAGGGTTAG-3′ [SEQ ID NO:1] which incorporates the repeating (TTAGGG) [SEQ ID NO:4].

Conveniently, the telomerase target sequence [SEQ ID NO:1] is fused at its 5′ end to a surface-linked spacer (or anchor) sequence [SEQ ID NO:2] comprising

| | |
|---|---|
| 5′-AATCCGTCGAGCAGAGTT-3′. | [SEQ ID NO: 2] |

The combined telomerase recognition sequence [SEQ ID NO:1] and the surface-linked spacer sequence [SEQ ID NO:2] is referred to as the spacer-telomerase recognition sequence [SEQ ID NO:3]:

[SEQ ID NO: 3]
5′-AATCCGTCGAGCAGAGTTAGGGTTAGGGTTAGGGTTAGGGTT
AG-3′.

Conveniently, the telomerase recognition sequence is immobilized via a thiol linkage. For example, a suitable linker is represented in SEQ ID NO:5:

[SEQ ID NO: 5]

5′SH(SCH₂)₆-TTTTTTAATCCGTCGAGCAGAGTTAGGGTTAG.

The present invention extends to any telomerase recognition sequence which is a substrate for human telomerase. In a particular embodiment, the telomerase recognition sequence is a human or non-human telomerase recognition sequence. Examples of non-human telomerase sequences include those from non-human primates, livestock animals and laboratory test animals such as from mice, rats, guinea pigs, hamsters, pigs or monkeys.

The TBT may also employ other solid supports including micropatterned surfaces, glass surfaces and supports, quartz crystal microbalance supports, microarrays, porous alumina supports, silica surface supports, nanoparticles, patterned polymer brushes, poly(ethylene glycol) brushes, membranes. The TBT may also be conducted on alternative systems such as nanoparticle amplified surface plasmon resonance (SPR) and BIAcore systems.

The present invention is particularly exemplified with respect to the use of biotin labeling of DNA. The biotin moiety on a dUTP is incorporated into the telomerase extended sequence. The biotin serves as a specific binding site to a reagent such as streptavidin-horseradish peroxidase (HRP), avidin-HRP or neutravidin-HRP that acts as a biocatalytic label in the presence of H₂O₂.

However, other labels may also be employed as long as an exogenous agent is added to visualize the label or in order to get a detectable signal. Hence, for example, a fluorescent, phosphorescent, chemiluminescent or radioactive label may be incorporated into the extended telomerase recognition sequence provided in order to maximize the resulting signal, an exogenous enhancer and/or signalling-facilitating agent is added. Alternative labels include but are not limited to biotin-dUTP, phycoerythrin-dUTP, fluorescein-dUTP and [α-³²P]-dUTP including all possible isomers thereof. The dNTPs include dATP and dGTP. Enzyme based and chemical detection assays may also be employed.

Accordingly, this aspect contemplates a method of detecting cells from a subject exhibiting telomerase activity said method comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the present of dNTP, including labeled dNTPs to thereby incorporate the label within the elongated primer;
ii) collecting the beads using a non-rotating magnet, washing the beads and contacting the washed beads with a signal-facilitating agent in order to maximize the signal produced from the label; and
iii) subjecting the resulting mixture to detection means to read the intensity of the signal,
   wherein the level of intensity of the signal compares to a control such as not containing telomerase-containing cells or a known data set provides the level of telomerase activity and the number of putative telomerase-exhibiting cells.

Conveniently, step (ii) or part thereof is conducted automatically or semi-automatically, such as in the machine which reads the luminescence intensity.

Although the control is generally a sample not containing a particular cell extract, it may equally be a sample not containing telomerase activity or labeled, dNTPs or other component required for operation of the assay. The control may also be a known data set of values which correlate to cell numbers.

It is important to note that the aspect of obtaining the cells and contacting an extract these with magnetic particles carrying an oligonucleotide primer which is a substrate for telomerase and incubating the particles in the presence of dNTPs to enable telomerase-mediated primer elongation occurs in the absence of any rotation of the beads. It is considered that the rotation of the beads is not required for acceleration of the kinetics of the reaction or would not accelerate the kinetics of the reaction. Hence, sensitivity of the TBT assay is not dependent on the rate of transport of anolytes or other substances that participate in the assay.

The assay of the present invention is also applicable to the detection of telomerase in a cell for research purposes, to determine the health status of the cell or to assess the ability for compounds to inhibit or enhance telomerase activity. This is applicable in all vertebrates, non-vertebrates and plants. In relation to vertebrates and in the case of cancer or an inflammatory condition, the condition may be diagnosed by removing tissue from a subject such as a human in order to screen for the presence of cancer cells or inflammatory cells. In addition, the presence of telomeres of a particular length may be required for proliferation of stem cells such as haematopoietic stem cells. This is important for blood transfusion such as in leukemia subjects. Blood samples may be screened for stem cells having particular telomerase activity which indicates a capacity for the stem cells to proliferate and differentiate into leukocytes and other cells of a hemopoietic lineage. For example, this method may be employed to monitor the success of stem cell mobilization by cytokines such as G-CSF, GM-CSF or other drugs. Thus this method may be used to augment and/or replace other methodologies used to monitor stem cells in peripheral blood or bone marrow.

Alternatively, tissue samples may be taken during treatment of a known cancer or inflammatory condition in order to evaluate the success or progress or otherwise of a treatment protocol or therapeutic regime. Such a regime may then be adjusted as necessary.

Accordingly, another aspect of the present invention provides a method for monitoring a treatment protocol such as for cancer or inflammation from a subject undergoing a treatment, said method comprising:
i) obtaining a sample of cells from said subject, contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture detection means to read the intensity of the luminescence,
   wherein the level of intensity of luminescence compared to a negative or positive control provides the level of telomerase activity wherein an increase in telomerase activity or a stabilization of telomerase activity is an indicator that the treatment protocol is not adversely affecting the subject.

Again, any of the steps but in particular step (iii) above may be conducted automatically.

The assay may also be used to screen for chemotherapeutic agents which reduce telomerase activity. Reference to a “chemotherapeutic agent” includes a chemical compound, immunological compound, natural product or sRNAi complex or a product of an introduced viral vector.

Accordingly, another aspect of the present invention contemplates a method for assessing the activity of a cytotoxic agent, said method comprising:
i) adding a putative cytotoxic agent to a culture of cancer cells;
ii) contacting magnetic particles carrying an oligonucleotide primer which is a substrate for telomerase with a cellular extract from the cancer cells and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
iii) contacting the magnetic particles with streptavidin-horseradish peroxidase;
iv) collecting the beads using a non-rotating magnet, washing the beads and contacting he washed beads with luminol and an enhancer and exogenous H₂O₂ to generate luminescence; and
v) subjecting the resulting mixture to detection means to read the intensity of luminescence,
   wherein the level of intensity of luminescence in the presence of the cytotoxic agent compared to a control such as not containing a cytotoxic agent provides the level of cytotoxicity of the agent.

The present invention further provides chemotherapeutic agents identified by the subject method as well as pharmaceutical compositions comprising same.

Generally, the subject being tested is a human. However, the present invention extends to any animal subject, and in particular a mammalian subject including primates (e.g. gorillas, marmosets, chimpanzees, monkeys), livestock animals (e.g. sheep, cattle, pigs, horses, goats), laboratory test animals (e.g. mice, rats, rabbits, guinea pigs, hamsters), companion animals (e.g. cats, dogs) and wild animals. Non-vertebrate mammals and plants are also contemplated by the present invention.

In the method of the present invention, the presence of the incorporated label is determined by the information of a signal, e.g. electrical signal, color signal or light emission. The sensing member is such that it can sense the signal, generally following a chemical or electronic signal. When the signal is emission of light the detector is a light detector.

When the signal is electrical, it results from the transfer of electrons between an electrode and an electron transfer chain, where the label is a member of that electron transfer chain.

Electrodes suitable for use in the method of the subject invention are made of or coated with conducting or semi-conducting materials, for example, gold, platinum, palladium, silver, carbon, copper and indium tin oxide.

Reference herein to “luminescence” includes chemiluminescence, bioluminescence, crystalloluminescence, electroluminescence, cathodoluminescence, photoluminescence, phosphorescence, fluorescence, sonoluminescence, thermoluminescence or triboluminescence.

Detecting means for the analysis of any type of signal, whether an electrical signal, color signal or light emission, are well known in the art.

The assay of the present invention is also applicable to the simultaneous or sequential detection of more than one label such as occurs during a multiplexing assay. In one example, multiple labels may be employed for different patient samples or from the same patient at different times or after different treatments. In such a case, the magnetic particles carry more than one label (either on the same magnetic particle or on different magnetic particles). In order for simultaneous detection to take place, the assay conditions are those that would allow the simultaneous formation of reaction signals that are distinguishable for each label. Accordingly, the presence of one label leads to a reaction signal of one type (e.g. light emission) while the presence of another label leads to a reaction signal of another type (e.g. emission of light in a different spectrum). Alternatively, the detection of the more than one label is achieved in sequence, such that after one assay is performed, the magnetic particles are collected, washed and provided with different assay conditions for the detection of another label. In such case, the reaction signal may be the same, provided that in each assay the reaction signal would be obtained solely in connection with the presence of a single label.

In the diagnostic methods of the invention, the assay is conducted to determine whether an elevated level of telomerase is present. The phrase “elevated level” means that the absolute level of telomerase activity in the particular cell is elevated compared to normal somatic cells in that individual or compared to normal somatic cells in other individuals not suffering from a disease condition. Generally, any detectable level of telomerase activity is considered elevated in cells from normal, post-natal human somatic tissue. Although telomerase activity is present in germline cells and low levels of telomerase activity can be detected in stem cells and certain hematopoietic stem cells, such cells do not present problems for the practitioner of the present method unless these cells are part of blood or tissue being transplanted. In that case (e.g. during a blood transfusion), stem cells with telomerase activity is desirable to ensure an ability to differentiate and proliferate. Germline cells can be readily distinguished and/or separated from human somatic tissue samples, and the telomerase activity present in stem cells and certain hematopoietic cells is present at such low levels that the few such cells present in somatic tissue samples will not create false positive signals from a telomerase activity assay. The detection of telomerase activity in somatic cells is indicative of the presence of immortal cells, such as certain types of cancer cells or inflammatory cells and can be used to make that determination even when the cells would be classified as non-cancerous or non-inflammatory pathology. Thus, the method of the present invention allows cancerous conditions to be detected with increased confidence before cells become visibly cancerous.

The diagnostic tests of the present invention can also be carried out in conjunction with other diagnostic tests. In some instances, such combination tests can provide useful information regarding the progression of a disease, although the present method for testing for telomerase activity provides much useful information in this regard. When the present method is used, for example, to detect the presence of cancer cells in a patient sample, the presence of telomerase activity can be used to determine where a patient is at in the course of progression of the disease, whether a particular tumor is likely to invade adjoining tissue or metastasize to a distant location and wether an occurrence of cancer is likely to recur. Tests that may provide additional information in conjunction with the present method include diagnostic tests for the estrogen receptor, progesterone receptor, DNA ploidy, fraction of cells in S-phase, nodal status, Her-2/neu gene products, p53, p16, p21, ras; EGF receptor, A33 (colon specific antigen) [Catimel et al, J. Biol. Chem 271(41):25664-25670, 1996], NY-ESO-1 (cancer testes antigen) [Chen et al, Proc. Natl. Acad. Sci. USA 94(5):1914-1918, 1997] or other oncogenes.

As indicated above, the TBT of the present invention is also useful for assaying for stem cells such as embryonic stem cells. In particular, TBT can be used to assess the therapeutic involvement of stem cells in disease conditions such as Parkinson's disease, heart disease, diabetes, arthritis, blood disease, osteoporosis, organ transplantation and spinal cord injury. The TBT is useful for monitoring the engraftment of stem cells or stem cell-derived tissue and to monitor the lifespan or state of differentiation of stem cells.

The present invention also provides kits for performing the diagnostic method of the present invention. Such kits can be prepared from readily available materials and reagents and can come in a variety of embodiments. For example, such kits can comprise any one or more of the following materials: reaction tubes, buffers, detergent, oligonucleotide telomerase substrates, control reagents, hydrogen peroxide and instructions. An especially preferred kit of the subject invention comprises a reaction tube in which is placed a telomerase substrate and dNTPs and biotylated dUTPs. A wide variety of kits and components can be prepared according to the present invention, depending upon the intended user of the kit and the particular needs of the user.

The present invention further contemplates the use of an assay which comprises:
i) obtaining a sample of cells from a subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads, optionally automatically, with luminol and an enhancer in the presence of exogenously added H₂O₂ to generate luminescence; and
iv) subjecting the resulting mixture detection means to read the intensity of the luminescence;
in the generation of a diagnostic protocol to detect cancer in a subject.

The present invention is further described by the following non-limiting Examples.

### EXAMPLE

The following examples are included to demonstrate particular embodiments of the invention. It should be appreciated by those of skill in the art that the techniques disclosed in the examples which follow represent techniques discovered by the inventors to function well in the practice of the invention, and thus can be considered to constitute particular modes for its practice. However, those of skill in the art should, in light of the present disclosure, appreciate that many changes can be made in the specific embodiments which are disclosed and still a like or similar result may be obtained without departing from the spirit and scope of the invention.

### Example 1

### Telomerase Luminescence Assay

This example describes the experimental protocols for a highly sensitive and selective biosensor assay, using luminescence as readout, to measure quantitatively telomerase in exfoliated tumor cells in the urine of bladder cancer patients or the stools from patients with colon cancer. Briefly, this assay uses superparamagnetic beads functionalized using thiol coupling to a nucleotide primer that contains the recognition sequence for telomerase. These beads (Biobeads) are incubated with tumor cell extracts, containing telomerase, in the presence of a nucleotide mixture that includes biotinylated-dUTP. Telomerase-induced elongation of the primers proceeds, with the incorporation of biotin-labeling. A number of biotin molecules are incorporated resulting in signal amplification. Avidin-Horseradish Peroxidase (HRP) is added which binds with high affinity (10⁻¹⁵M) to the incorporated biotin. The Biobeads are then well washed, which minimizes contamination by other potentially interfering substances in the bulk biological sample (the magnetic particles can be efficiently trapped using a magnet) and transferred to a 96 well plate in a BMG Luminometer. Hydrogen peroxide, luminol and a chemical enhancer are added, optionally automatically and the luminescence signal detected.

This protocol conveniently uses a Kingfisher Magnetic Particle Processor to aid assay automation. A BMG Luminometer (BMG, Lattech, Germany) is also used. The chemiluminescence reader may also be modified to allow for automation such as the addition of enhancer, luminol and/or H₂O₂. It can be set up in multiple plate format.

The use of the magnetic particles is designed to facilitate automation. The particles themselves can be picked up and manipulated using a magnet: the Kingfisher Magnetic Particle Processor (Thermo Corporation, USA), for example, mixes and moves magnetic particles with electromagnetic magnetic rods covered by disposable tips which prevent cross contamination. The Kingfisher Software allows custom-made protocols to be designed for specific applications. During the initial steps, beads are collected, buffer, reaction mixture and samples added and mixed. Manual intervention is required to transfer the plates to a Labnet Shaking Incubator to elevate the temperature (37 C, 30 min) to drive the telomerase extension reaction. Use of the Kingfisher 96, which has built in temperature control, obviates this requirement. After incubation the plate is then transferred back to the Kingfisher for the final addition of the streptavidin HRP followed by the rigorous washing steps, which are essential to maintain the constant low background observed in the assay. Finally, the magnetic beads carrying the extended oligonucleotide are transferred into a Nunc 96 well LumiNunc plate: these plates have minimum autoluminescence. The plate is then transferred manually to a BMG Fluorostar Luminometer (BMG Labtech, Germany): this fully automated microplate based multi-detection reader, which is equipped with injectors that deliver reagent at the point of measurement, can be programmed for the addition of luminol (Pierce SuperSignal ELISA Femto Substrate) and peroxide. The same instrument can be used for fluorescence detection. Transfer between workstations (Kingfisher and BMG) can be further automated using robotic transfer (e.g. Zymark Twister, Beckman Sagian). Likewise, alternative technologies can be substituted for the Kingfisher (e.g. Beckman Biomek, Bruker Daltronics ClinProt Robot) or the BMG Fluorostar (e.g. Molecular Devices LMax II).

### Experimental Protocol for Kingfisher Magnetic Particle Processor

Add the following to Plate A1 (and B1 if using two plates):
Row A—100 μl Elongation Buffer
Row B—specified volume of Reaction Mix (total volume once telomerase added is 50 μl)
Row B—add telomerase enzyme extract
Row A—finally add 10 μl of oligo coupled dynal beads (bead stock of 30 mg/ml) to the 100 μl of elongation buffer in each well—ensure sufficient mixing.

Place plate(s) into Kingfisher instrument and slide in a comb(s) coverslip to protect the magnets.

Select program—“Telomerase Assay”
Hit “Start” twice.
When Kingfisher pauses—it will instruct ‘Incubate at 37 C’
Take plate(s) out, cover Rows A & B with Nescofilm (Registered Trade Mark) to ensure plate sealed well.
Place plate(s) into Labnet shaking incubator (settings: Temp=37 C; Time=30 min; RPM=12)

Following 30 minutes incubation, remove Nescofilm (Registered Trade Mark) and fill the remaining rows as follows:
Row C—100 μl 1% w/v SDS/10 mM HEPES
Row D—100 μl 1% w/v SDS/10 mM HEPES
Rowe E—100 μl Working Buffer/Tween
Row F—100 μl Working Buffer/Tween
Row G—100 μl Working Buffer/Tween
Row H—100 μl Working Buffer/Tween

Place plate(s) back into Kingfisher and press “Start” (method will continue).

Set up plate A2 (and B2 if using 2 plates)

Add the following to Plate A2 (B2):
Row A—100 μl Working Buffer/Tween
Row B—500 μl 0.5 ug/ml Strept HRP (KPL) in working buffer/tween)
Row C—100 μl Working Buffer
Row D—100 μl Working Buffer
Row E—100 μl Working Buffer
Row F—100 μl Working Buffer
Row G—100 μl Working Buffer
Row H—50 μl Working Buffer

When Kingfisher pauses and instructs ‘Change plates’
Swap plates over

Press “Start” and the method will resume.

Prime BMG FluoroStar Luminometer with Luminol in pump A and peroxide in pump B—set up plate template (APL assay—well mode) and volumes (50 ul of each). Luminometer gain to be set to 2000.

At end of method, machine will beep continually, press end.

Take plate(s) from Kingfisher and transfer Row H into a NUNC white 96 well plate and place in BMG FluoroStar Luminometer for reading.

### Buffers

### Elongation Buffer

### 20 mM Tris-HCl

### 1.5 mM MgCl₂

### 63 mM KCl

### 1 mM EGTA

### 1 mM EDTA

### 150 mM NaCl

### 0.05% Tween20

### SDS Buffer

### 0.1% w/w SDS

### 10 mm HEPES

### Working Buffer

### 0.1M Tris, pH 7.4

### 0.1M KCl

### (0.05% v/v Tween 20)

### Reaction Mix

1× Elongation buffer

### 0.25% w/v BSA

### 12.5 uM B-dUTP

18.75 uM dAdG
Telomerase (or extract containing telomerase activity)

### MilliQ H₂O

Remove two aliquots of sample to be assayed (typically 1-5 μl).
Heat one aliquot at 95° C. for 20 minutes to heat inactivate the sample then place on ice.
Aliquot reagents into 96 well Kingfisher plate and place in Kingfisher particle processor (see protocol in separate document).
Wash Dynal magnetic beads coupled with the specific telomerase oligonucleotide sequence (Oligo-bead) in 100 μL elongation buffer (1×) for 2 min.
Transfer beads into 504 of the reaction mix (elongation buffer, 12.5 μM Biotin-dUTP, 0.25% w/v BSA, 18.75 μM dAdG) and sample.
Manually transfer plate to the heater/shaker instrument.
Incubate oligo-beads with reaction mix for 30 minutes at 37° C. to enable elongation of the oligo by the enzyme in the sample.
Manually transfer plate back to Kingfisher instrument.
Wash oligo-beads ×2 with 100 μL 1% w/v SDS/1 mM HEPES for 2 minutes at RT.
Wash oligo-beads ×5 with 100 μL elongation buffer (1×) for 2 minutes at RT.
Incubate oligo-beads with 50 μl of 1 ug/ml Streptavidin-HRP for 30 minutes at RT.
Wash oligo-beads ×5 with 100 μL of working buffer (1M Tris-HCl, 1M KCl, pH 7.4) for 2 minutes at RT.
Resuspend oligo-beads in a final volume of 50 μL working buffer and transfer sample into a white luminescence plate.
Place white plate into luminometer for luminescence results (50 μL of luminol and 50 uL of peroxide are added automatically by the instrument).

### Example 2

### Thiol Coupling of Target Sequence to Beads

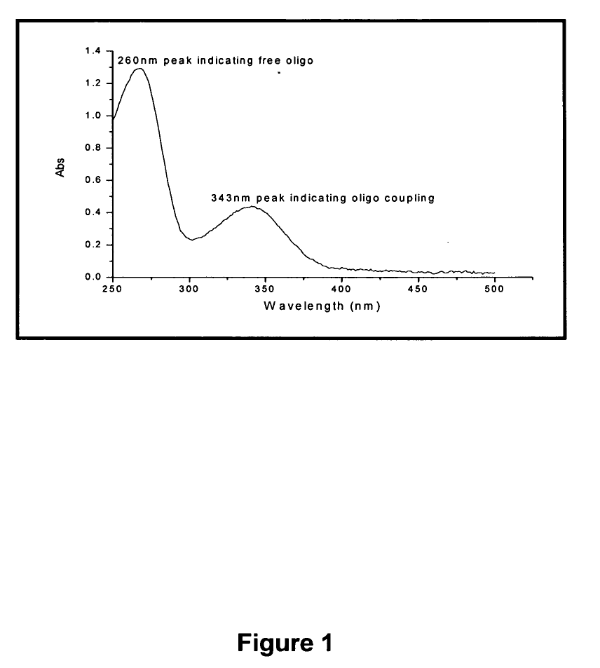

A target sequence for telomerase, with a 5″ cysteine for thiol coupling (5′SH(CH₂)₆-TTTTTTAATCCGTCGAGCAGAGTTAGGGTTAGGGTTAG [SEQ ID NO:5]) was conjugated to magnetic beads using the heterobifunctional crosslinker Sulfo-LC-SPDP (Pierce). The oligo is reduced using 50 mM trialkylphosphine (tris(2-carboxyethyl) phosphine) (TCEP) for 2 hr at RT. The reduced oligo is purified from the TCEP by size exclusion chromatography on a Superpose 12 HPLC column (Amersham). The reduced oligo is then incubated with Sulfo-LC-SPDP modified magnetic beads overnight at 4° C. The conjugation is monitored via an increase in the 343 nm absorbance reading (see FIG. 1).

### Example 3

### Telomerase Biosensor Test (TBT)

A telomerase assay was conducted as follows:

The telomerase target sequence [SEQ ID NO:1] was synthesized using a bead surface-binding oligonucleotide [SEQ ID NO:2] and the combined sequence [SEQ ID NO:3] immobilized to a Dynal (Dynal Invitrogen Corporation, 9099 North Deerbrook Trail, Brown Deer, Wis., USA 53223). Immobilization was via a cysteine residue binding to the 5′ end of SEQ ID NO:3.

Cells were obtained containing putative cancer cells and lysed with CHAPS buffer [0.5% v/v CHAPS, 10 mM Tris, 1 mM MgCl₂, 1 mM EGTA and 10% v/v glycerol with 1 protease inhibitor tablet (Compete Mini, Roche) per 10 ml]. The lysed cell extract was then added to the magnetic beads with dNTPs and biotinylated dUTP. Streptavidin-HRP was then added. After incubation, the beads were collected using a magnet without rotation and washed. The beads were then transformed to a 96 well plate. Luminol and an enhancer were added together with hydrogen peroxide. Luminescence was then read.

### Example 4

### Sensitivity of Assay

LIM1215 carcinoma cells (Whitehead et al, J Natl Cancer Inst 74(4):749-765, 1985) were counted and aliquots removed containing from 100 to 1000 cells and assayed for telomerase. The results are shown in FIG. 2. The graph shows that the sensitivity is as low as one cell. Samples comprising 10⁶ cells or greater were also assayed with good detection of telomerase activity.

### Example 5

### Sample Preparation

A list of cancers and the sampling technique is provided below. The list is only exemplary of the types of cancers which can be detected by the methods of the present invention.

Sample workups for some of these are included under Item 11. Examples of the type of clinical sample on which the TBT would be used are indicated for each cancer.

bladder cancer: sedimented cells in urine, bladder washings;
urogenital tract cancer: renal pelvic washings, bladder washings;
renal cancer: renal pelvic washings, bladder washings;
colon cancer: exfoliated faecal epithelial cells, endoscopic biopsy specimens;
leukemia: bone marrow and peripheral blood;
melanoma: peripheral blood, fine needle aspirates;
skin cancer: biopsy, peripheral blood, fine needle aspirates;
lung cancer: Bronchial alveolar lavage, bronchial brushings and washings, sputum, scrapings and smears, fine needle aspirates, biopsies and tissue sections;
prostate cancer: fine needle aspirates, sedimented cells in urine;
head and neck cancer: scrapings and smears;
lymph nodes: fine needle aspirates;
pancreas: fine needle aspirates;
salivary gland: fine needle aspirates;
breast: fine needle aspirates, nipple discharge;
liver: fine needle aspirates;
thyroid: fine needle aspirates;
brain cancer: cerebrospinal fluid; and
cervical, vaginal and ovarian cancer: smears, peritoneal washings.

### Example 6

### Superficial Bladder Cancer Sample

Samples of 5 μl comprising cells were assayed.

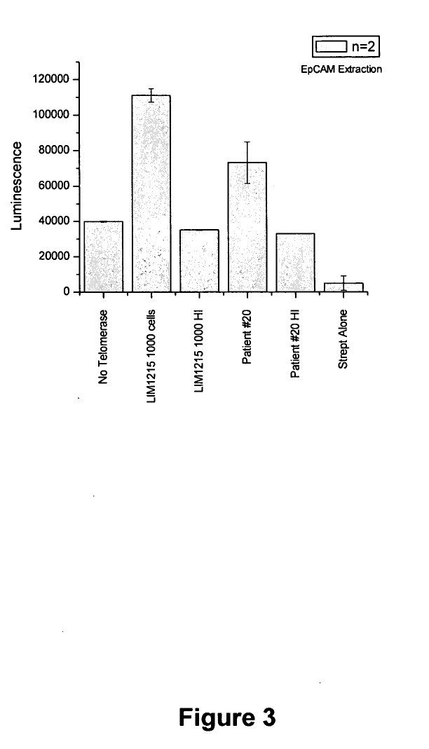

The LIM1215 colon cancer cell line (Whitehead et al, 1985 supra) is a positive control. Note reduction is signal following heat inactivation (HI). Ep-CAM beads were used to separate the cancer cells from activated lymphocytes during the sample workup. The results are shown in FIG. 3.

### Example 7

### Invasive bladder Cancer sample

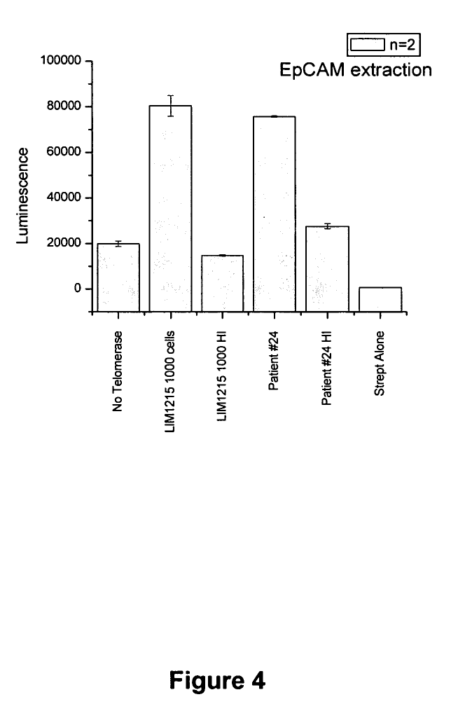

Samples of 1 μl comprising cells were assayed. The results are shown in FIG. 4.

### Example 8

### Comparison of Telomerase Assay with the TRAP Method

Using telomerase assay as described in Example 3, a comparison was made with the TRAP assay (Hess et al, 2002 supra). The results are shown in Table 3. The telomerase assay appears to be more sensitive than the TRAP assay. See also Example 18.

TABLE 3
Clinical Samples assayed using the TBT assay

| Cancer Type | Telomerase assay | TRAP Results |
|---|---|---|
| Superficial | Positive | Negative |
| Superficial | Positive | Negative |
| Superficial (CIS) | Positive | NT |
| Invasive | Positive | Negative |
| Invasive | Positive | NT |
| Normal | Negative | NT |
| Normal | Negative | NT |

NT = Not Tested

### Example 9

### Isolation of Colonocytes from Fecal Samples

Determine weight of fecal sample.
Vigorously vortex sample in 50 mL/g PUCK's dispersing buffer with additives (See Puck's buffer recipe).
Filter slurry through 100 μm membrane and collect flow through.
Filter collect liquid through 60 μm membrane and collect flow through.
Centrifuge flow through for 10 minutes at 1000 g at 4° C.
Resuspend cell pellet in 2.5 mL of PUCK's dispersing buffer with antibiotics.
Layer over a discontinuous gradient of 7.5 mL Percoll and centrifuge at 20 000×g for 20 minutes @ 4° C. (fixed angle rotor).
Collect the cell fraction and make up to 10 mL with PBS containing 1% v/v FCS and 0.6% w/v sodium citrate.
Centrifuge for 10 minutes at 1000 g at 4° C.
Resuspend in PBS containing 1% v/v FCS and 0.6% w/v sodium citrate (2 ml).
Aliquot 40 μl (1×10⁷) Dynal EpCAM beads into 2 tubes and wash ×2 in PBS/0.1% w/v BSA.
Aliquot 2 mL sample into the 2 tubes (1 mL each) containing 40 μl EpCAM beads and incubate for 30 at 4° C. with rotation.
Place tubes in magnet and remove S/N (Keep for cytospin).
Resuspend in PBS/0.1% w/v BSA (200 μL)—Pool both tubes into one and put into magnet to remove the 400 μl of supernatant.
Repeat 200 uL PBS/0.1% w/v BSA wash ×2.
Add 200 uL of CHAPS lysis buffer.
Lyse cells by passing through a fine needle.
Incubate lysates on ice for 30 minutes.
Spin at 10,000 g for 20 minutes at 4° C.
Aliquot S/N and snap freeze in LN2.
Snap freeze remaining cell pellet.

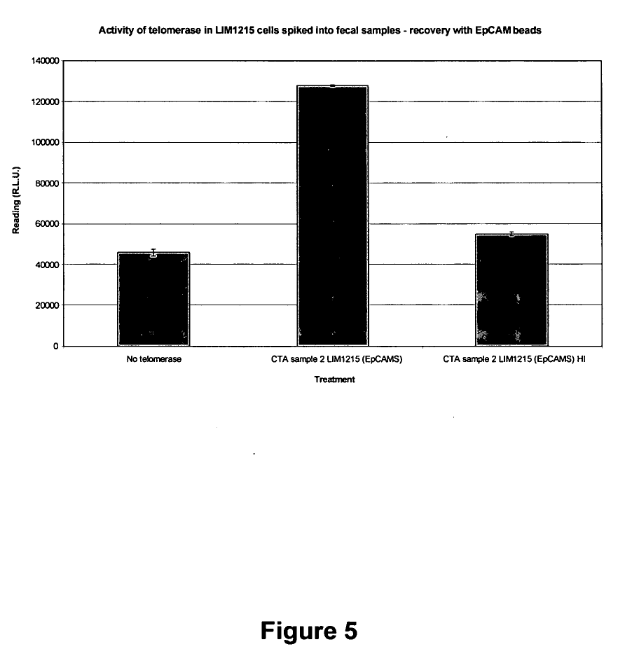

The assay results are shown in FIG. 5.

### Example 10

### Sensitivity of TBT

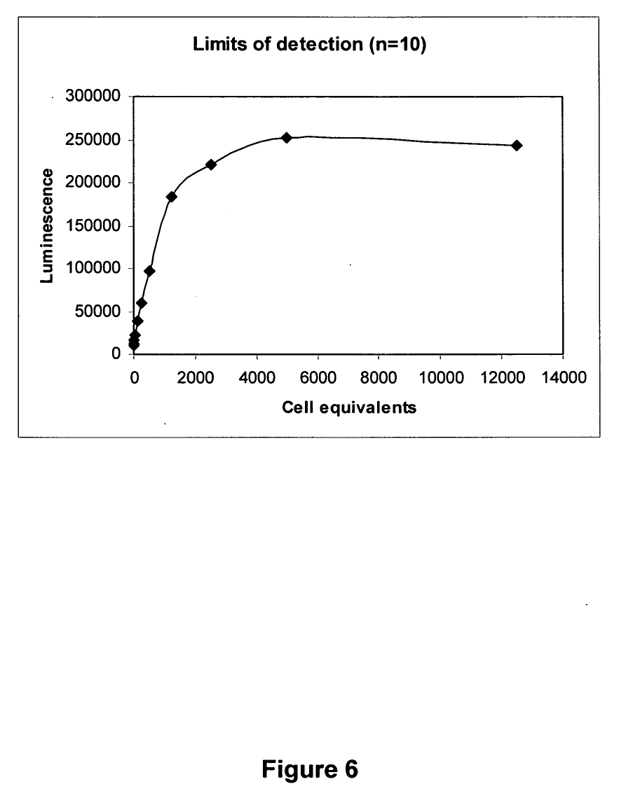

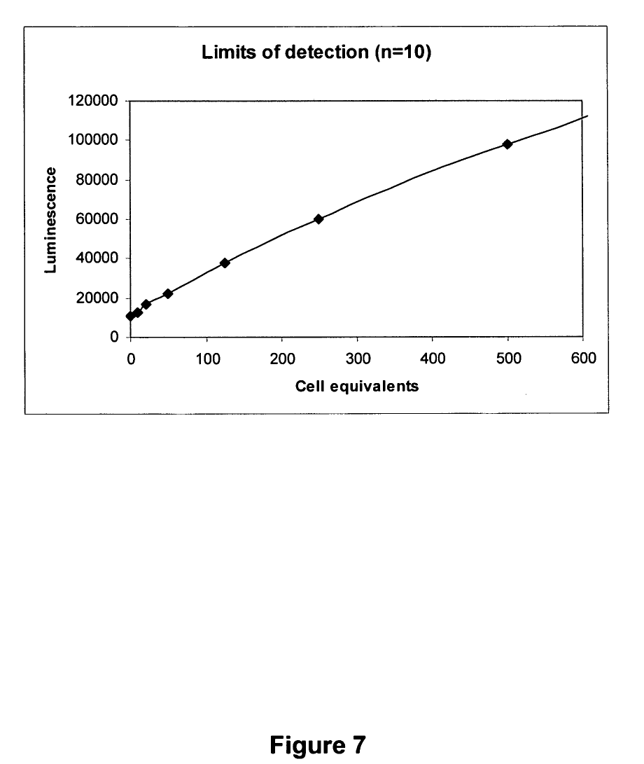

HEK293T (Graham et al, J Gen Virol 36:59-74, 1977) tumor cell lysate was assayed for telomerase over a broad range of lysate concentrations, from 10-1250 cell equivalents (CE). The relationship between the TBT result (luminescence signal) and the lysate concentration is linear up to approximately 1250 CE, after which the TBT signal begins to plateau. The TBT response relationship is linear at concentrations of cells expected in the urine of bladder cancer patients. The results are shown in FIGS. 6 and 7.

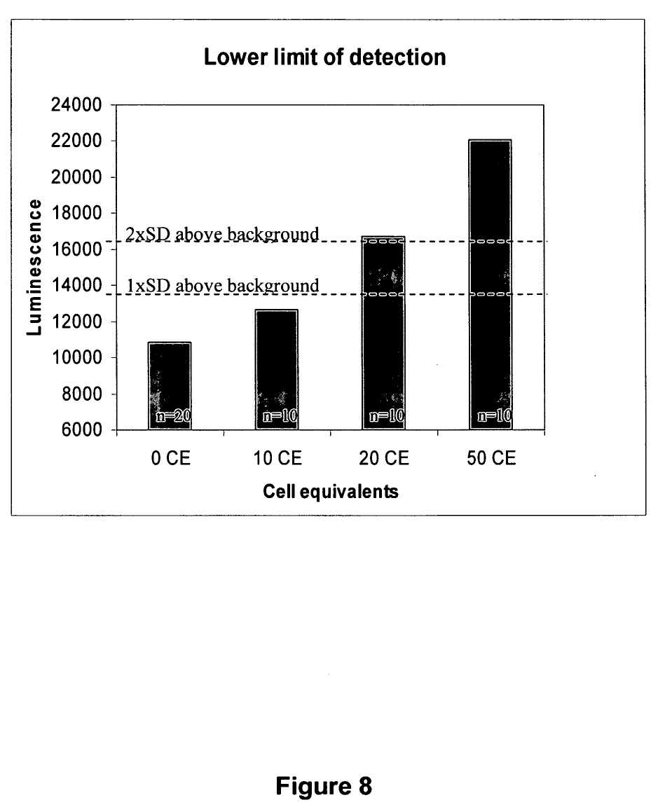

The TBT assay performs at very low cell concentrations. Statistical evaluation of the lower limit of detection revealed that the minimum number of cells detectable with the TBT assay is as few as 20 CE. The results are shown in FIG. 8. The TBT test can detect positive signals from very few numbers of telomerase-expressing cells. It, therefore, has the capability of detecting very small numbers of exfoliated tumor cells in urine.

### Example 11

### Intra-Assay Reproducibility

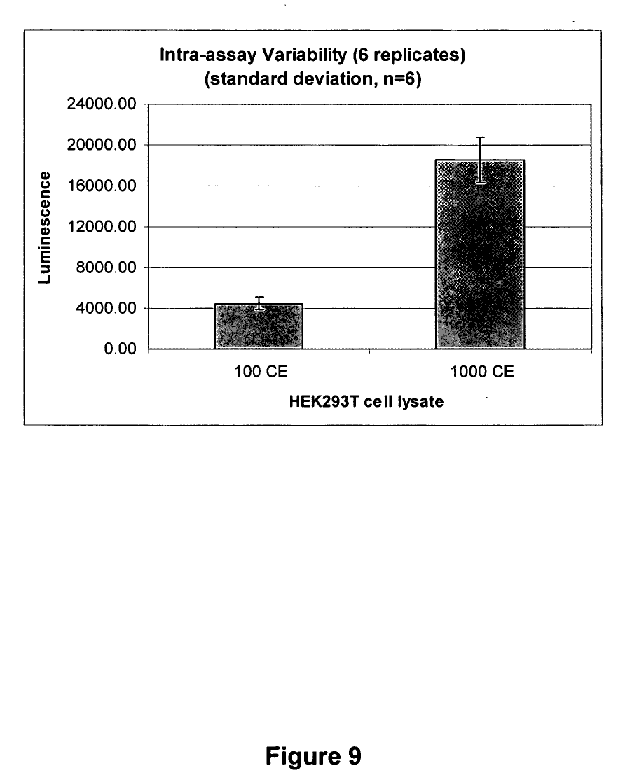

The within assay reproducibility of the TBT test was assessed by measuring the telomerase activity of two different concentrations, 100 CE and 1000 CE, of HEK293T tumor cells. The level of variability, between six replicate samples at each concentration, within the TBT assay was approximately 5%. The results are shown in FIG. 9.

### Example 12

### Inter-Assay Reproducibility

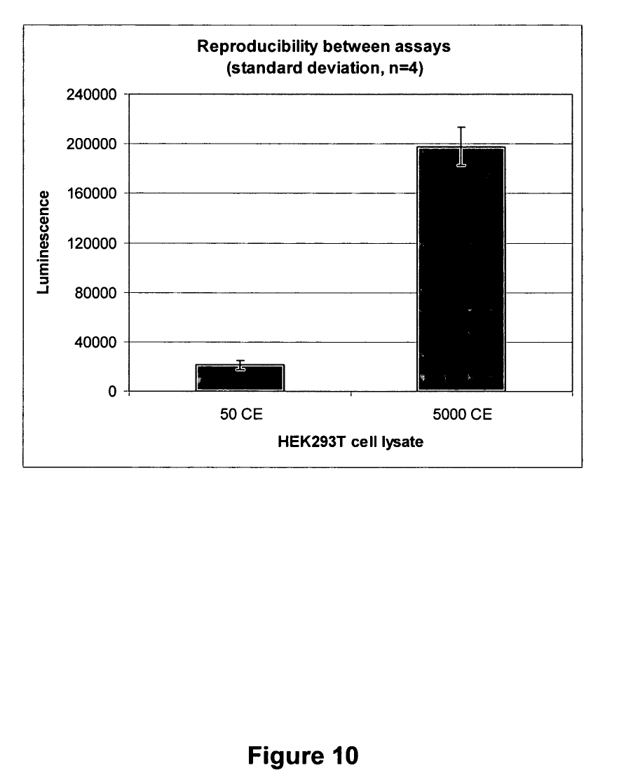

The between assay reproducibility of the TBT test was assessed by measuring the telomerase activity of two different concentrations of HEK293T tumor cells, 50 CE and 5000 CE. The assay was performed on four separate occasions on different days. The level of between assay variability for each concentration ranged from 6-9%. The results are shown in FIG. 10.

### Example 13

### Specificity of Assay

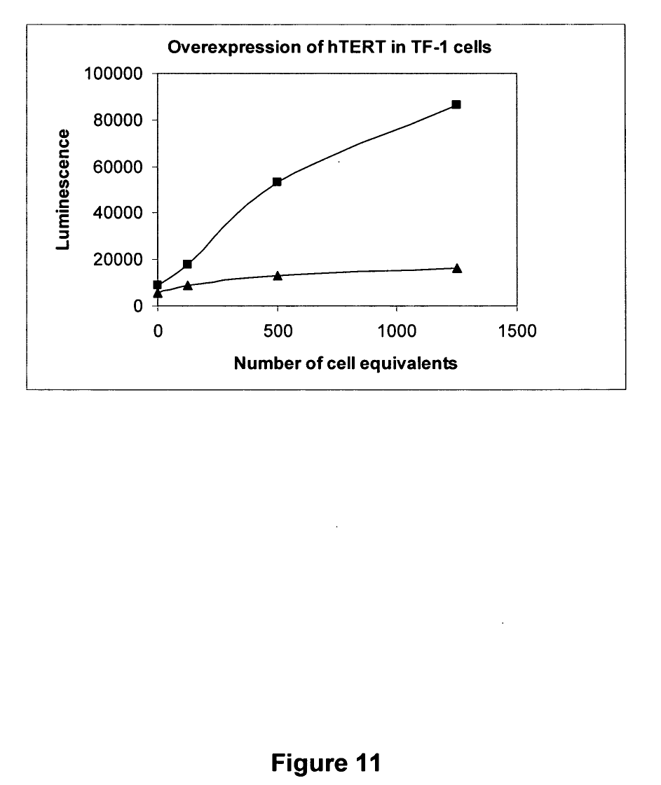

The specificity of the TBT test was determined by measuring the telomerase activity of tumor cells overexpressing the human telomerase reverse transcriptase (hTERT). Telomerase activity was measured in the TF-1 human erythroleukaemia cell line (Kitamura et al, Blood 73(2):375-380, 1989) containing retroviral vectors expressing the human telomerase reverse transcriptase [hTERT] (Li et al, Leukemia 20:1270-1278, 2006). The results are shown in FIG. 11.

### Example 14

### Measurement of Telomerase Activity in Urine Samples

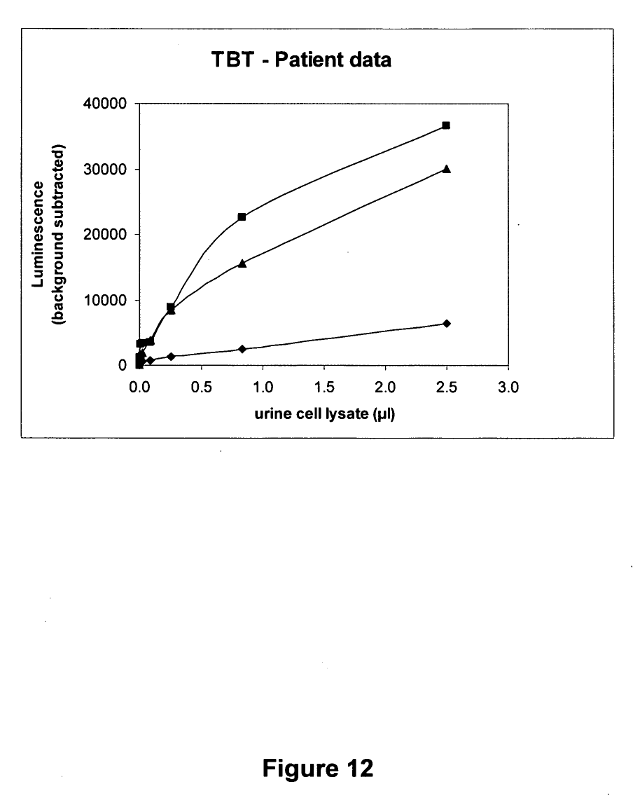

The TBT test was used to measure telomerase activity in cells, isolated from the urine of bladder cancer patients, in cell lysate concentrations ranging from 0 μl to 2.5 μl of lysate. Telomerase activity was measured in urine cell lysates from three patients previously showing a positive TBT result, two of which having a high TBT result (Patient #3, TBT ratio 6.70 and Patient #12, TBT ratio 6.38) and one patient having a low TBT result (Patient #31, TBT ratio 1.59). The results are shown in FIG. 12.

### Example 15

### Bladder Cancer Monitoring

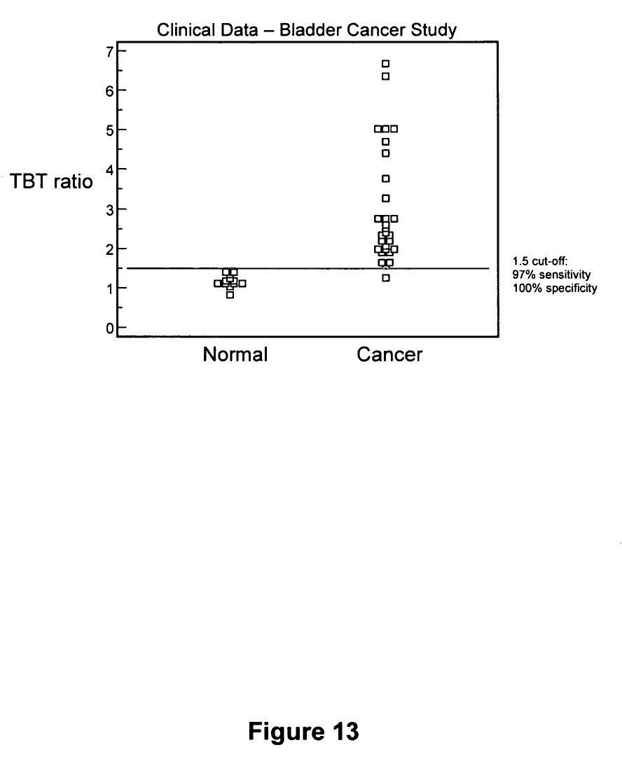

The TBT test was used to measure telomerase activity in cells, isolated from the urine of bladder cancer patients and normal subjects. A positive TBT test signal is defined as a signal >1.5-fold higher in magnitude than the background signal. The results are shown in FIG. 13 and Table 4.

TABLE 4
Clinical data - summary
Ratio (Test/HI - heat inactivated)

| Patient Group | Mean ± SEM | N |
|---|---|---|
| Normal | 1.20 ± 0.05 | 12 |
| Cancer | 3.02 ± 0.27 | 29 |

### TBT Data

Mean data given above represents averaged TBT results. Aside from the relation to the “Cut-Off” value, there appears to be little correlation between the magnitude of the TBT result and the stage and severity of bladder cancer.

### Example 16

### Leukemia

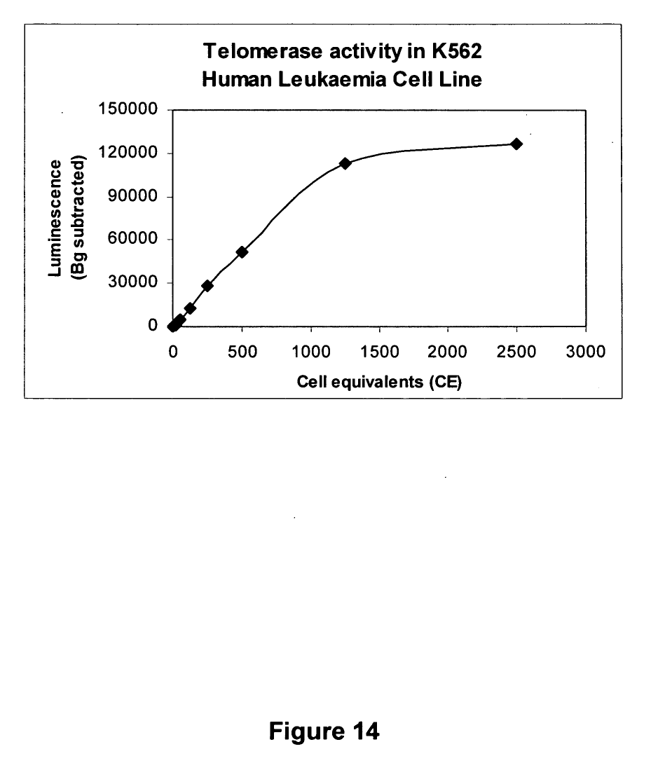

The TBT test was used to measure telomerase activity in the human leukemia cell line K562 (Lozzio and Lozzio, Blood 45:321-334, 1975). The TBT assay is sensitive for the detection of telomerase activity in leukemia cells, is quantitative and relatively simple to perform compared to existing methods for measuring telomerase in leukemia cells. The results are shown in FIG. 14.

### Example 17

### Umbilical Cord Blood Stem Cells

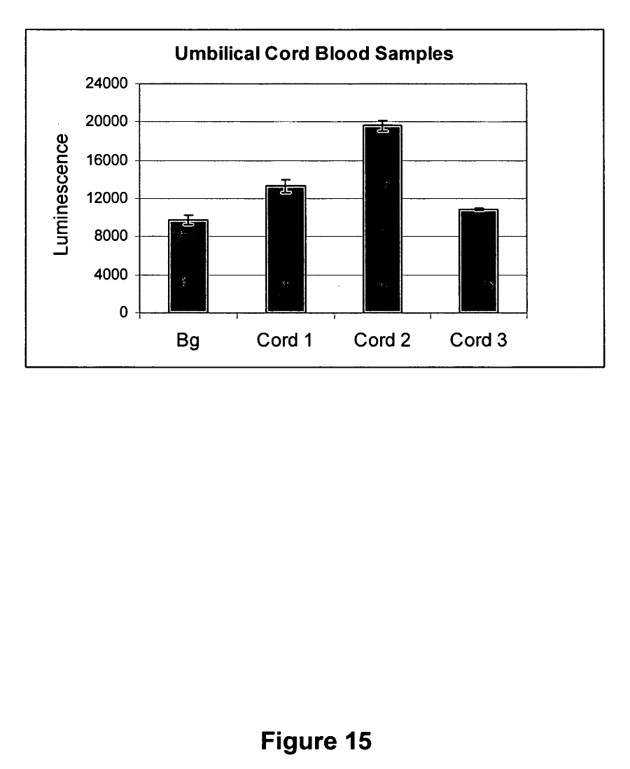

The TBT test was used to measure telomerase activity in umbilical cord blood stem cells. The TBT assay was sensitive for the detection of telomerase activity in umbilical cord blood stem cells in all three cord blood samples, using 1000 CE. The results are shown in FIG. 15.

### Example 18

### Comparative Assays

The assay features of the present invention and the standard TRAP assay are compared. A summary of the comparative features is provided in Table 5. The comparison highlights the improved efficacy of the TBT compared to the TRAP assay.

TABLE 5
Comparison with standard TRAP

| Assay Feature | TBT | Reasons | TRAP | Reasons |
|---|---|---|---|---|
| Time frame | <2½ hours | Employs test tubes | 1 day | Employs PCR thermo- |
| | | and plates, simple | | cycling, running of |
| | | pipetting steps, and | | gels, and complicated |
| | | magnetic beads | | image analysis |
| Operating | High | When fully | Low | Limited by gel |
| throughput | | automated, can run | | running and |
| | | 96 wells (or more) | | complicated analysis |
| | | every 2½ hours. | | |
| Routine Use | High | Uses standard | Not | The need to run PCR |
| | | laboratory | possible | products on gels and |
| | | equipment. Highly | | the sophisticated |
| | | amenable to | | image analysis |
| | | automation on | | prevents routine |
| | | typical pathology | | clinical utility |
| | | lab robotic systems | | |
| Automation | High | Magnetic bead- | Not | Non-standard assay |
| | | based method | possible | methodology (gels, |
| | | suitable for liquid handling | | image analysis) |
| | | robotics readily available | | prevents automation |
| | | in routine pathology | | |
| | | laboratories | | |
| Sensitivity - | High (<10 | Positive signals are | High | Optimal conditions for |
| research use | cells) | detected below 10 | (variable) | PCR difficult to attain |
| | | HEK293T or | | |
| | | LIM1215 cells | | |
| Sensitivity - | High (93- | Positive signals | Low | Very poor sensitivity |
| clinical samples | 97%) | detected from | | in our hands, possibly |
| | | clinical samples | | due to PCR |
| | | close to cut-off | | contamination issues. |
| Interference | Low | Sample workup | High | PCR reaction easily |
| | | purifies cells of | | “poisoned” by |
| | | interest and | | interfering substances |
| | | removes them from | | in clinical samples; |
| | | interfering | | eg; blood components |
| | | substances. | | (haemoglobin). |
| Robustness | High | Magnetic beads are | Low | Typical PCR issues |
| | | very stable. | | such as cross- |
| | | Telomerase | | contamination, and |
| | | reaction robust. | | interference of Taq |
| | | Chemiluminescence | | polymerase by |
| | | widely used in | | endogenous |
| | | clinical labs. | | substances. PCR |
| | | | | introduces errors in |
| | | | | the quantity and ratio |
| | | | | of extension products. |
| Quantitation | Quantitative | Luminescence | Semi- | Logarithmic laddering |
| | | measurement can | quantitative | of the product |
| | | be defined per cell | at best | necessitates |
| | | number (equivalent | | complicated image |
| | | lysate) | | analysis of DNA on |
| | | | | gels. |
| False results | Rare, if any | | Common | Both false negative |
| | | | | and false positive |
| | | | | results are common |
| | | | | due to PCR inhibitors, |
| | | | | over-sensitivity or |
| | | | | PCR contamination |
| Ease of operation | Good | Easy pipetting | No. Time | Need to run gels. |
| | | procedures, can be | consuming | Difficult analysis. |
| | | fully automated. | and | Some TRAP assays |
| | | | requires | use radioactivity for |
| | | | skilled | detection. |
| | | | operator | |
| Costs | Low | Instrumentation | High | Expensive kits |
| | | simple, reagents | | |
| | | relatively | | |
| | | inexpensive. | | |

### Example 19

### Monitoring Telomerase Activity in Conjunction with Telomerase Therapeutics

The TBT is of benefit in selecting and monitoring patients who are subject to therapies that target telomerase activity and components of the telomerase complex. Such applications include vaccines against telomerase components as may be used in the treatment of cancers or autoimmune or hyper-proliferative disorders.

Similarly, the TBT can be used to monitor the reactivation of telomerase activity as part of therapies such as stem cell activation in tissue regeneration, replacement, repair and restoration such as skin or other organs. Other contexts include the activation of stem cell activity in bone marrow, neurogenic zone of the adult brain, and the reactivation of T lymphocytes in HIV patients. Other contexts include gastrointestinal and respiratory tract recovery following damage such as that produced by chemotherapies or radiotherapies.

Similarly, the TBT is applicable to monitor the efficacy of telomerase inhibitors in the context of drug development in the laboratory setting, in animal models and in patients.

Similarly, the TBT is useful to monitor the maintenance of stem and progenitor cell activity in tissues reconstituted with embryonic stem cell-derived cells and tissues where there is a need to achieve short or long term tissue replacement.

### Example 20

### Repetitions of the Telomerase Repeat Sequence

Design of the telomerase-specific oligonucleotide template attached to the magnetic bead is critical for maximizing sensitivity of the TBT assay. The minimal recognition DNA sequence for base-pairing between the RNA component of telomerase and the telomere end is 9 bases—TAGGGTTAG, however, multiple repeats of this sequence more accurately depict the nature of chromosome telomere ends and the scanning nature of enzymes used to achieve accurate base-pair recognition. Telomerase templates include those ranging from 1.5-3 hexameric repeats.

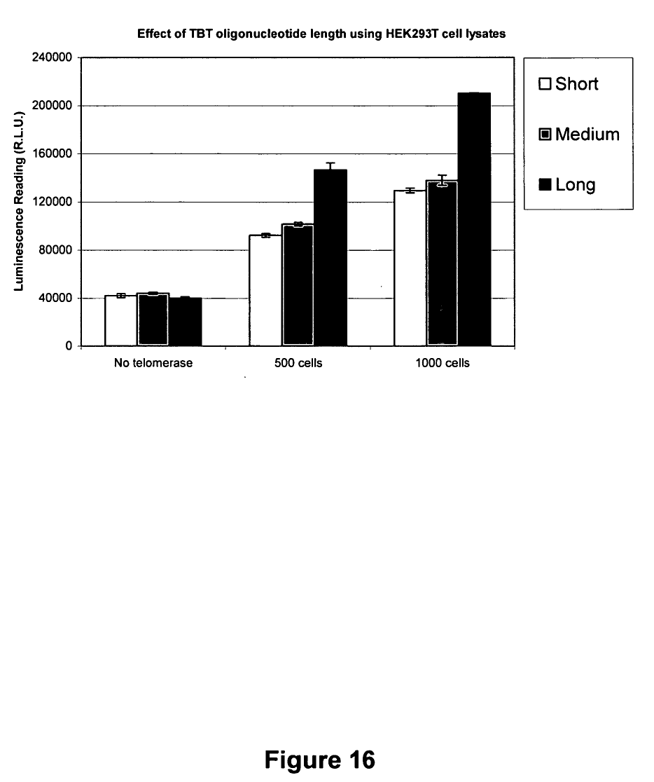

Three forms of the oligonucleotide were tested. A short version containing a partial (0.5) repeat, a slightly longer version containing 2.5 repeats, and a longer version with 3.5 repeats. The longer version provided better absolute signal relative to the background signal (no telomerase extract). This increased dynamic range is likely to translate into increased sensitivity of the assay. The results are shown in FIG. 16.

The oligonucleotides tested were as follows:

Short:
(SEQ ID NO: 6)
5′SH—(CH2)6-TTTTTTAATCCGTCGAGCAGAGTT
Medium:
(SEQ ID NO: 7)
5′SH—(CH2)6-TTTTTTAATCCGTCGAGCAGAGTTAGGGTTAGGG
TTAG
Long:
(SEQ ID NO: 8)
5′SH—(CH2)6-TTTTTTAATCCGTCGAGCAGAGTTAGGGTTAGG
GTTAGG GTTAGGGTTAG

The length of oligonucleotide may also be important for shelf-life stability. Reaction beads are stored in the presence of EDTA and EGTA which bind/sequester free metal ions. Metal ions are essential co-factors for enzymes that degrade nucleic acids and therefore their removal protects the oligonucleotides from degradation.

### Example 21

### Automation

The TBT assay is highly amenable to automation because it uses standard magnetic bead technology. Magnetic bead-based liquid handling robotic systems are used commonly in routine pathology laboratories for a variety of applications. The TBT assay can be easily adapted to a variety of such systems and is not machine-dependent.

Automation of the TBT assay puts it at a distinct advantage compared to other techniques such as TRAP and the assay described in PCT/IL01/00808 (WO 02/20838). The latter employs a rotating electromagnet and cannot be readily automated. It is not suitable for routine pathology lab use. TRAP in its original form requires that PCR products are run on electrophoresis gel and subsequently analysed by imaging, hence it is not suitable for high throughput automation.

### Example 22

### Cell Capture Beads

Any cell-specific antibodies, receptors or mimetics can be used for purification of cells of interest for telomerase activity measurement. These include for example:

Anti-EGF receptor (for tumour cells);
Anti-CD34 (stem cells);
Anti-CD45 (common leukocyte antigen);
Anti-CD19 (pan-B-cell antigen) CD4 and CD8 (lymphocytes);
Anti-BerEP4 (pan-epithelial cell surface antigen); and
Anti-A33 (Colonic epithelial antigen)

Cells can also be purified or isolated by other methods such as continuous or non-continuous ficoll gradients for isolation of peripheral blood mononuclear cells (PBMC).

### Example 23

### Sample Preparation Method and Workups

The TBT test can be used for the detection of malignant cells in relation to many different cancers. Typical clinical samples that may be analysed using the TBT test include, but are not restricted to, the following:

Bronchial alveolar lavage, bronchial brushings and washings, sputum, scrapings, smears for the detection of neoplasms in the bronchial tree, lung cancer, head and neck cancer.

Fine needle aspirates, biopsies and tissue sections for the detection of malignant cells in the lung, lymph nodes, pancreas, salivary gland, breast, liver, thyroid, and in prostate cancer.

Sedimented cells in urine, renal pelvic washings, bladder washings for the detection of prostate cancer, bladder cancer, urogenital tract cancer, and renal cancer.

Blood for the detection of melanoma and cancers of the haematopoietic system.

Body cavity fluids (pleural fluid, peritoneal fluid, pericardial fluid, peritoneal washings, gutter washings) for the detection of malignant neoplasms.

Cerebrospinal fluid for the detection of malignant cells in the CSF.

Endoscopic biopsy specimens for the detection of cancer of the gastrointestinal tract.

Faecal specimens for the detection of malignant cells in colon cancer and other cancers of the gastrointestinal tract.

Nipple Discharge: for the detection of breast cancer and cancers causing nipple discharge.

PAP TestTM/PAP smears (Cervical/Vaginal Screening) for the detection of cervical, vaginal and ovarian cancer. May also be used for the detection of certain infectious and inflammatory conditions.

Skin (TZanck Smear) for vesicular diseases secondary to herpes virus infections (Herpes Simplex virus and Varicella-Zoster virus).

In the case of bladder cancer, tumor epithelial cells are isolated by selective capture from urine using epithelial cell-specific antibodies attached to magnetic beads.

### Example 24

### Urine Processing Procedure—Sample Workup

All steps are performed on ice to prevent the non-specific attachment of cells to the Dynal beads.
1. Urine is collected (at least 50 mls) and kept on ice. The urine is transferred to a 50 ml tube. If there is more than 50 mls, the urine is divided into 2 equal volumes in the 50 ml tubes and each processed as below.
2. Sample is centrifuged at 750 g for 5 minutes at 4° C. Supernatant is discarded into a beaker containing a HazTab.
3. Pellet is resuspended in 10 ml PBS (pH 7.4), supplemented with 0.1% w/v BSA and a protease inhibitor tablet (thereafter referred to as wash buffer).
4. Sample is centrifuged at 750 g for 5 minutes at 4° C. Supernatant is discarded into the beaker with the HazTab.
5. Washing step is repeated (steps 4-5).
6. During step 5, the Epithelial Enrich Cellection Dynal beads are washed once with 100 μl wash buffer (using the Dynal magnetic trap).
7. Following centrifugation, the pellet is re-suspended in 1 ml of wash buffer and transferred to a 1.5 ml eppendorf tube.
8. Washed beads are added to the washed urine cells from Step 5. For pellets that are less than 1 mm in diameter, 25 μl of beads are used. For pellets between 1-2 mm, 30 μl of beads are used. For anything larger than 2 mm, 40 μl of beads are used.
9. The beads and urine cells are mixed gently for 30 minutes at 4° C. with rotation (60 r.p.m.).
10. Samples are centrifuged (Capsule Tomy HF120) for 30 sec to ensure that no beads or buffer is left in the lid of the eppendorf tube.
11. Tubes are placed in the Dynal Magnetic Trap (Dynal MPC-S), and the supernatant carefully transferred to a fresh 1.5 ml Eppendorf tube using a Gilson P1000 pipette: Supernatant is centrifuged at 13,000 r.p.m. in a Hereaus Biofuge for 5 minutes at 4° C. Supernatant is removed and cells in the pellet lysed (this contains cells that have not bound to the Epithelial Enrich Cellection Dynal beads). This fraction may contain activated lymphocytes and should be stored separately as a frozen cell pellet (−70° C.) for subsequent analysis, if required.
12. Beads from Step 11 are washed by re-suspending in 1 ml wash buffer and then the supernatant is removed using the Dynal magnetic trap as described above. This supernatant is discarded.
13. CHAPS lysis buffer (100 μl) is added to the Dynal beads bound to the epithelial cancer cells.
14. Cells are lysed by pipetting up and down at least 10 times using a Gilson P200 pipette.
15. Lysates are incubated on ice for 30 minutes.
16. Lysates are centrifuged at 13,000 r.p.m. in a Hereaus Biofuge for 5 minutes at 4° C.
17. Beads are removed by place tubes in the magnetic trap.
18. Supernatants (˜30 μl) are aliquotted into each of 3 tubes and the pellet discarded.
19. Lysates are snap-frozen on dry ice for 5 minutes and transferred to −70° C. refrigerator.

### Example 25

### Sample Workup for Exfoliated Colonocytes from Faecal Samples of Colon Cancer Patients

Faecal samples are collected under informed consent from patents with clinically proven colorectal cancer. Samples are collected at home and transported immediately to the laboratory (less than 2 hours) where aliquots (2 g) are dispersed in Puck's saline with antibiotics (500 U/L penicillin, 500 mg/L Streptomycin-sulphate, 1.25 mg/L amphotericin B and 50 mg/L gentamicin). The faecal slurry is filtered sequentially through 100 μm and 60 μm membranes (Nylon/Net membrane filters, Millipore, Australia) to remove large debris before being centrifuged at 400 g for 10 minutes at 4° C. The pellet is washed twice with PBS containing 1% v/v FCS and 0.6% w/v sodium citrate, followed by recovery of epithelial cells using 40 μl Epithelial Enrich CELLection (Trade Mark) Dynabeads. The cells are incubated with the Dynabeads for 30 min at 4° C. and the supernatant then removed using the Dynal Magnetic Particle Processor. The cells attached to the magnetic beads are washed 3 times with PBS containing 0.1% w/v BSA before lysis with 200 μl CHAPS lysis buffer. The resulting supernatant is snap frozen in liquid nitrogen and stored at −70° C.

### Example 26

### Sample Workup for Umbilical Cord Stem Cells: (Enrichment of Lineage-Negative Cells)

Human umbilical cord blood (UCB) is collected in sterile bottles containing an anticoagulant citrate buffer and processed within 18 hours of collection. To deplete red blood cells, UCB is diluted 1:2 with Dulbecco's phosphate-buffered saline, and red blood cells agglutinated at room temperature using 1% w/v Hespan (DuPont Pharma, Wilmington, Del.). Residual red blood cells are lysed with 0.17 mM NH4Cl, 10 mM Tris-Cl at pH 7.2, 0.25 mM EDTA. Lineage-negative (Lin-) cells are isolated by depletion of cells expressing glycophorin A, CD3, CD2, CD56, CD24, CD19, CD66b, CD14, and CD16 using the StemSep kit (Stem Cell Technologies, Vancouver, British Columbia, Canada) according to kit instructions. The percentage of CD34+ cells in the resulting Lin-fraction ranges from 63% to 82%.

### Example 27

### Sample Workup for Leukemia Cells

Cells for diagnosis and analysis of leukemia patients are isolated from bone marrow or peripheral blood. Ten-ml human bone marrow aspirates, taken from the iliac crest of normal donors, are diluted 1:1 with phosphate-buffered saline and centrifuged at 900 g for 10 minutes at room temperature. The washed cells are resuspended in PBS to a final volume of 10 ml and layered over an equal volume of 1.073 g/ml Percoll solution. After centrifugation at 900 g for 30 minutes, the mononuclear cells (MNCs) are recovered from the gradient interface and washed with PBS. Percoll-fractionated MNCs or non-fractionated bone marrow cells are suspended in PBS for analysis. MNCs are isolated from buffy coats of peripheral blood by Ficoll-Paque density gradient centrifugation and washed in PBS.

### Example 28

### Receiver Operating Characteristic Curve

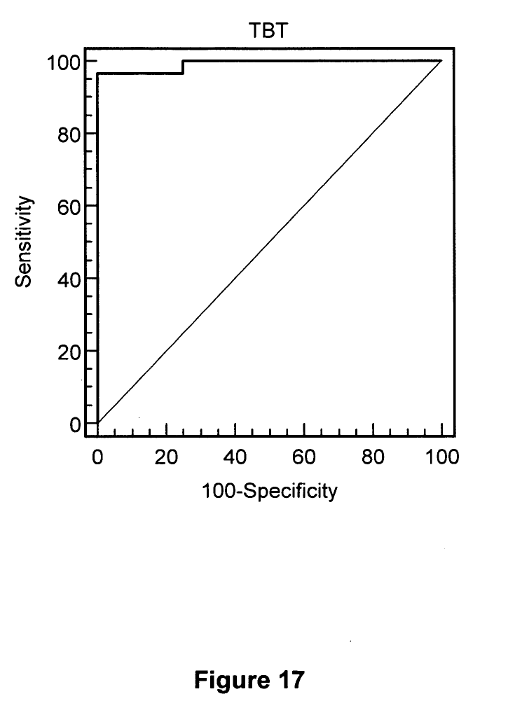

FIG. 17 shows a “Receiver Operating Characteristic” curve (ROC curve) evidencing the sensitivity of the TBT test in detecting bladder cancer.

The ROC curve depicts the pattern of sensitivities and specificities observed in the clinical study when the performance of the TBT test is evaluated at different diagnostic thresholds. The overall diagnostic performance of the TBT test is judged by the position of the ROC line. Poor tests have lines close to the rising diagonal, whereas lines for perfect tests rise steeply and pass close to the top left hand corner, where both the sensitivity and specificity are 1. The ROC line for the TBT closely approaches the line for a perfect diagnostic test.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Catimel et al, J. Biol. Chem. 271(41):25664-25670, 1996
Chen et al, Proc. Natl. Acad. Sci. USA 94(5):1914-1918, 1997
Graham et al, J Gen Virol 36:59-74, 1977
Hess et al, Clin. Chem. 48:18-24, 2002
Hiyama et al, Cancer Lett. 194:221-223, 2003
Kim et al, Science 266:2011-2015, 1994
Kitamura et al, Blood 73(2):375-380, 1989
Li et al, Leukemia 20:1270-1278, 2006
Lozzio and Lozzio, Blood 45:321-334, 1975
Shay et al, Hum. Mole. Gen. 10:667-685, 2001
Weizmann et al, Chem. Bio. 5:943-948, 2004
Whitehead et al, J Natl Cancer Inst 74(4):749-765, 1985

1. A method for detecting cells from a subject exhibiting telomerase activity, said method comprising:
i) obtaining a sample of cells from said subject and contacting magnetic particles carrying an oligonucleotide primer which is a substrate for telomerase with a cellular extract from said sample and incubating said magnetic particles and said cell extract together for a time and under conditions sufficient for telomerase-mediated elongation of the oligonucleotide primer to occur in the presence of NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to a detection means to read the intensity of the luminescence,
wherein the level of intensity of said luminescence compared to a control not containing the cells to be detected or to a known data set provides the level of telomerase activity and the number of cells.

2. The method of claim 1, wherein the oligonucleotide primer comprises the sequence (XₙTTAGGYₘ)ₒ wherein:
X is a nucleotide selected from the group consisting of A, T, G and C;
Y is a nucleotide selected from the group consisting of A, T, G and C;
n is 0 or 1;
m is 0 or 1; and
o is from about 1 to about 400.

3. The method of claim 2, wherein the oligonucleotide primer is selected from the group consisting of SEQ ID NO:1 and SEQ ID NO 3.

4. The method of claim 1 wherein said subject is selected from the group consisting of a human, a non-human, a vertebrate and a plant.

5. The method of claim 1, wherein said sample of cells are selected from the group consisting of cancer cells, inflammatory cells, and stem cells.

6. The method of claim 1 wherein said oligonucleotide primer is immobilized to the magnetic particles via a thiol linkage.

7. The method of claim 1 wherein the addition of an agent selected from the group consisting of luminal, enhancer and H₂O₂ is automatic or semi-automatic.

8. The method of claim 1 wherein the cancer is selected from the group consisting of any cancerous condition, any malignant condition, any pre-cancerous condition, a myeloma, any lymphoma and any other proliferative disorder involving neoplastic cells.

9. The method of claim 8 wherein the cancer is selected from the group consisting of breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors, Wilm's tumor, adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's-cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed-cell, null-cell, t-cell, t-cell chronic, htlv-ii-associated, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nscic), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (scic), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), sertoli cell tumor, synovioma, testicular cancer, thymus cancer, thyroid cancer, transitional-cell-cancer-(bladder), transitional-cell-cancer-(renal-pelvis−/−ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

10. The method of claim 5 wherein the inflammatory cells are involved in a condition selected from the group consisting of acne, angina, arthritis, aspiration pneumonia, disease, empyema, gastroenteritis, inflammation, intestinal flu, nec, necrotizing enterocolitis, pelvic inflammatory disease, pharyngitis, pid, pleurisy, raw throat, redness, rubor, sore throat, stomach flu and urinary tract infections, chronic inflammatory demyelinating polyneuropathy, chronic inflammatory demyelinating polyradiculoneuropathy, chronic inflammatory demyelinating polyneuropathy and chronic inflammatory demyelinating polyradiculoneuropathy.

11. The method of claim 1, wherein said cells are selectively purified or enriched prior to detecting the level of telomerase activity.

12. The method of claim 11, wherein said purification or enrichment includes incubating the cells in magnetic beads located with cell-specific antibodies followed by washing to remove potentially interfering substances.

13. A method for assessing the activity of a cytotoxic agent, said method comprising:
i) adding a putative cytotoxic agent to a culture of cancer cells;
ii) contacting magnetic particles carrying an oligonucleotide primer with a cellular extract from said cancer cells and incubating said magnetic particles and said cell extract together for a time and under conditions sufficient for telomerase-mediated elongation of the oligonucleotide primer to occur in the presence of NTPs and biotinylated UTPs to thereby incorporate biotin within the elongated primer;
iii) contacting the magnetic particles with streptavidin-horseradish peroxidase;
iv) collecting the beads using a non-rotating magnet, washing the beads and contacting the washed beads with luminol and an enhancer and exogenous H₂O₂ to generate luminescence; and
v) subjecting the resulting mixture to a detection means to read the intensity of luminescence,
wherein the level of intensity of luminescence in the presence of the cytotoxic agent compared to a control such as not containing a cytotoxic agent provides the level of cytotoxicity of the agent.

14. The method of claim 13 wherein said oligonucleotide primer is immobilized to the magnetic particles via a thiol linkage.

15. The method of claim 13 wherein the addition of an agent selected from the group consisting of luminal, enhancer and H₂O₂ is automatic or semi-automatic.

16. The method of claim 13, wherein said cells are selectively purified or enriched prior to detecting the level of telomerase activity.

17. The method of claim 16, wherein the cells were purified or enriched by incubating the cells in magnetic beads located with cell-specific antibodies followed by washing to remove potentially interfering substances.

18. A method of detecting cancer in a subject comprising:
i) obtaining a sample of cells from said subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture to a detection means to read the intensity of the luminescence,
in the generation of a diagnostic protocol to detect cancer in a subject.

19. The method of claim 18, wherein said oligonucleotide primer is immobilized to the magnetic particles via a thiol linkage.

20. The method of claim 18, wherein the addition of an agent selected from the group consisting of luminal, enhancer and H₂O₂ is automatic or semi-automatic.

21. The method of claim 18, wherein said cells are selectively purified or enriched prior to detecting the level of telomerase activity.

22. The method of claim 21, wherein said cells are purified or enriched by incubating the cells in magnetic beads located with cell-specific antibodies followed by washing to remove potentially interfering substances.

23. A kit for detecting telomerase activity, said kit comprising in compartment form a first compartment comprising magnetic particles carrying an oligonucleotide primer which is a substrate for telomerase; a second compartment comprising reagents including hydrogen peroxide; a third compartment comprising dNTPs or biotinylated dNTPs and instructions for detecting said telomerase activity comprising:
i) obtaining a sample of cells from a subject and contacting magnetic particles carrying an oligonucleotide primer which is a substrate for telomerase with a cellular extract from said cell sample and incubating the magnetic particles and cell extract together for a time and under conditions sufficient for telomerase-mediated elongation of the oligonucleotide primer to occur in the presence of the NTPs and biotinylated UTPs to thereby incorporate biotin within the elongated primer;
ii) contacting the magnetic particles with streptavidin-horseradish peroxidase;
iii) collecting the beads using a non-rotating magnet, washing the beads and contacting the washed beads with luminol and an enhancer in the presence of exogenously added H₂O₂ to generate luminescence; and
iv) subjecting the resulting mixture detection means to read the intensity of the luminescence,
wherein the level of intensity of luminescence compared to a control not containing the cells to be detected or to a known data set provides the level of telomerase activity and the number of cells.

24. The kit of claim 23, wherein said level of telomerase activity is useful for detection of cancer or for assessing the cytotoxic potential of an anti-cancer agent.

25. The kit of claim 23, wherein the oligonucleotide primer comprises the sequence (XₙTTAGGYₘ)ₒ wherein:
X is a nucleotide selected from the group consisting of A, T, G and C;
Y is a nucleotide selected from the group consisting of A, T, G and C;
n is 0 or 1;
m is 0 or 1; and
o is from about 1 to about 400.

25. The kit of claim 23, wherein the oligonucleotide primer is selected from the group consisting of SEQ ID NO:1 and SEQ ID NO:3.

26. The kit of claim 23, wherein the oligonucleotide primer is immobilized to the magnetic particles via a thiol linkage.

27. The kit of claim 23, wherein the addition of an agent selected from the group consisting of luminol, enhancer and H₂O₂ is automatic or semi-automatic.

28. The kit of claim 24, wherein the cancer is selected from the group consisting of any cancerous condition, any malignant condition, a pre-cancerous condition, a myeloma, any lymphoma, and any proliferative disorder involving neoplastic cells.

29. The kit of claim 28, wherein the cancer is selected from the group consisting of breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors, Wilm's tumor, adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, branchioma, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumor, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, giant cell tumors, ganglioneuroma, glioma, glomangioma, granulosa cell tumor, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's-cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. b-cell, mixed-cell, null-cell, t-cell, t-cell chronic, htlv-ii-associated, lymphangiosarcoma, lymphocytic acute, lymphocytic chronic, mast-cell and myeloid), leukosarcoma, leydig cell tumor, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nscic), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (scic), small intestine cancer, soft tissue sarcoma, spinal cord tumors, squamous-cell-carcinoma-(skin), stomach cancer, synovial sarcoma, sarcoma (e.g. Ewing's experimental, Kaposi's and mast-cell sarcomas), sertoli cell tumor, synovioma, testicular cancer, thymus cancer, thyroid cancer, transitional-cell-cancer-(bladder), transitional-cell-cancer-(renal-pelvis−/−ureter), trophoblastic cancer, teratoma, theca cell tumor, thymoma, trophoblastic tumor, urethral cancer, urinary system cancer, uroplakins, uterine sarcoma, uterus cancer, vaginal cancer, vulva cancer, Waldenstrom's-macroglobulinemia and Wilms' tumor.

30. The kit of claim 23, further comprising magnetic particles having antibodies immobilized thereon directed to cells selected from the group consisting of cancer, leukemia, inflammatory and stem cells.
